(19) United States
(12) Patent Application Publication
(10) Pub. No.: US 2009/0053751 A1
(43) Pub. Date: Feb. 26, 2009

(54) Chemiluminescent Method and Device for Evaluating the In Vivo Functional State of Phagocytes

(76) Inventors: Moni Magrisso, Beer Sheva (IL); Robert S. Marks, Omer (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(21) Appl. No.: 11/885,519

(22) PCT Filed: Feb. 28, 2006
(86) PCT No.: PCT/IL2006/000272

§ 371 Date: Sep. 18, 2008

Related U.S. Application Data
(60) Provisional application No. 60/656,926, filed on Mar. 1, 2005.

(51) Int. Cl. C12Q 1/02; C12M 1/34

(52) U.S. Cl. 435/29; 435/288.7

(57) ABSTRACT

A method of assessing the in vivo state of phagocytes in a patient, possibly indicating diagnostically important states such as inflammation or infection, which method utilizes chemiluminescent (CL) light emitted during the reaction in vitro between a CL substrate and the reactive oxygen species (ROS) formed in a fluid sample obtained from the patient. The measurement is performed in two or more portions of the sample, with stimulated phagocytes affected by one or more priming agents which shift the functional state of the phagocytes, providing a plurality of measurements, which are analyzed so as to distinguish intracellular and extracellular contributions to the CL kinetics. The results are compared with a range of control measurements performed with patients suffering from various diagnostic conditions.

### FIELD OF THE INVENTION

The present invention relates to a method and a device for evaluating the in vivo functional state of phagocytes of a patient by in vitro measurements of chemiluminescent (CL) kinetics in a sample obtained from said patient. More particularly, the present invention relates to characterizing extracellular and intracellular contributions to said kinetics, wherein phagocytes respiratory burst is measured in a plurality of portions of said sample, said portions comprising different priming conditions. Calculated parameters indicate the conditions of said patient's immune system. The method also enables to assess an effect of a pharmacologic agent on phagocytes in vitro.

### BACKGROUND OF THE INVENTION

Human phagocytes play a key role in the innate immune response to infection. They act at inflammatory sites, which they reach after targeting and extravasation from the peripheral blood stream where they are normally present. Upon interaction with invading microorganisms or inflammatory mediators, they produce large amounts of toxic reactive oxygen species (ROS), such as superoxide anion and hydrogen peroxide, by activation of the NADPH-oxidase. The degree of activation as well as the subcellular localization of the toxic oxygen radicals generated is determined by the identity of the agonist and the cell-surface receptor involved in the activation process. This process, known as the “respiratory burst”, is responsible for the oxygen-dependent microbicidal activity of the polymorphonuclear leukocytes (PMNs) [Babior et al.: J. Clin. Invest. 52 (1973) 741-4]. Additionally, PMNs release from their cytoplasmic granules bactericidal products, such as bacterial permeability-increasing protein, lysozyme, lactoferrine, and defensins, which are responsible for the oxygen-independent killing of the microorganisms. Proteolytic and hydrolytic enzymes present in the same granules provide the digestion and degradation of the microorganism debris.

ROS produced by PMNs are normally used for elimination of invading microorganisms. Measuring various functions of PMNs becomes increasingly important in medical diagnosis and prognosis. Deficiencies in the first-line defense system create a high risk for infections that may even include septic complications. However, excessive production of such species may promote tissue injury, an important factor in the pathogenesis of many diseases [Malech et al.: N. Engl. J. Med. 317 (1987) 687-94]. Overactivated phagocytes may lead to autoaggresive damage of tissues, comprising at the local level, e.g., gout, rheumatoid arthritis, and emphysema, or at the systemic level multiple organ failure, systemic inflammatory response syndrome, and adult respiratory distress syndrome. PMNs circulate in a “priming state”, which is a state “pre-tuned for future tasks”, reflecting the organism's readiness for defense and, therefore, being of high predictive value [Maderazo et al.: J. Infect. Dis. 154 (1986) 471-7]. Attempts have been made to correlate the primed activity of circulating PMNs with the severity of disease and its outcome [Wakefield et al.: Arch. Surg. 128 (1993) 390-5]. However, this priming state is extremely sensitive, and can be substantially disturbed by cell isolation procedures usually preceding the functional tests. Therefore, whole-blood techniques that avoid cell separation are preferred [e.g., Kukovetz et al.: Redox Report 1 (1995) 247].

When granulocytes interact with soluble or particulate matter in the presence of luminol, the cells will respond and produce chemiluminescence (CL), a reaction linked to the bactericidal oxidative metabolism of the granulocytes. This makes it possible to measure the triggering of an oxidative burst in a small number of cells, such as those available from neonates [Mills et al.: Pediatrics. 63 (1979) 429-34] or from neutropenic subjects [Stevens et al.: Infect. Immun. 22 (1978) 41-51]. Since the requirements for the laboratory equipment are modest and since CL measurements are simple to perform, the technique has been increasingly used a) to follow disease activity or early infection—before antibodies are detectable; b) to evaluate immunomodulating activity of pharmacological products; c) to provide information about the interactions between phagocytes and biomaterials; d) to follow PMNs metabolic activity associated with microbicidal events; e) for screening granulocytes for defects in oxidative metabolism; and f) to provide information about the interaction between phagocytes and allergenic microbial and industrial pollutants.

The luminol amplified chemiluminescent reaction in neutrophils requires the presence of a peroxidase and oxygen metabolites produced by the NADPH-oxidase, wherein said peroxidase is usually myeloperoxidase (MPO) originating from azurophil granules. The result of an interaction between neutrophils and invading bacteria should be bacterial killing with minimal damage to the surrounding tissue components. This means that if a bacterium-neutrophil interaction leads to ingestion of the prey, the cellularly produced oxygen metabolites should be released inside the phagosome. If, however, the prey remains on the neutrophil surface, the metabolites have to be released extracellularly to reach the bacterium. The techniques commonly used to measure the production of reactive oxygen metabolites involve a large detector molecule that cannot reach the intracellular site [detcalf et al.: Laboratory manual of neutrophil function. Raven Press, New York, 1986]. Thus, with the use of these techniques, only oxidative metabolites released extracellularly are quantified. With the use of the luminol-amplified CL technique, however, the extracellular as well as intracellular events in a cellular response can be measured [Bender and van Epps: Infect. Immun. 41 (1983) 1062-70; Briheim et al., Infect. Immun. 45 (1984) 1-5]. The extracellular CL response can be separated from the intracellular one [Dahlgren: Inflammation 12 (1988) 335-49], utilizing the fact that the CL reaction, as peroxidase-dependent, is totally inhibited by azides, which are MPO inhibitors [Edwards J.: Clin. Lab. Immunol. 22 (1987) 35-9], and the fact that both H₂O₂ scavenger catalase and azide-insensitive horse reddish peroxidase (HRP) are large proteins that have no access to intracellular sites. Since the CL systems used for separate quantifications of intracellular and extracellular ROS production are different, direct quantitative comparison of extracellularly released ROS and intracellularly released ROS are impossible. Another problem during these measurements is the formation of cell sediment at the chamber bottom during the CL measurement. The matrix/erythrocyte layer between sediment-forming phagocytes and the photodetector absorbs and scatters the light produced by phagocytes thus decreasing the instrument sensitivity. It is therefore an object of the invention to provide a method of quantifying the ROS production by phagocytes, taking into account the extracellular and intracellular contributions, avoiding the drawbacks of existing methods.

Optical fiber-based biosensors have demonstrated their ability to detect biological entities with high sensitivity, due to the intimate coupling between the specific biological interactions and the fiber core with minimal signal losses [Marks et al.: Appl Biochem. Biotechnol. 89 (2000) 117-26]. Moreover, it has been shown that a silica surface stimulates circulating blood phagocytes to produce a CL pattern similar to the extracellular phase of the fMLP-induced pattern (fMLP stands for N-formyl-methionyl-leucyl-phenylalanine) [Tuomala et al.: Toxicol. Appl. Pharmacol. 118(2) (1993) 224-32]. It is therefore another object of the invention to provide a device for quantifying the ROS production by phagocytes, taking into account the extracellular and intracellular contributions, using the optical fiber-based biosensors.

The quantification of CL signal from human neutrophils has been found to be useful in the detection of genetic deficiencies, and studies of inflammatory diseases, infection, degenerative diseases, and cancer. The main findings involving genetic diseases are in the diagnosis of the neutrophil abnormalities (i.e. chronic granulomatous disease, and myeloperoxidase deficiency). Studies related to cell CL in inflammatory diseases include arthritis, exercise-induced asthma, and pollen-induced allergy; bacterial and viral infections have been followed using CL of human neutrophils; cellular CL has been employed also in research of diabetes, renal dialysis, and cancer (including leukemia).

U.S. Pat. No. 5,108,899 describes a method of evaluating the in vivo state of inflammation of a patient by measuring CL response of phagocytes. The method is based on assessing the total reactivity reserve of the phagocytes, i.e., on measuring the maximal CL response available in the phagocytes after priming in vitro. The method, however, does not enable to assess the relative contributions of intracellularly and extracellularly generated ROS to the total oxidative phagocyte response, thus losing a part of the information about the state of phagocytes that is potentially extractable from the CL signal. It is therefore still another object of this invention to provide a method for evaluating the in vivo state of phagocytes by analyzing the CL signal obtained in vitro, wherein both intracellularly and extracellularly generated ROS contribute to the information yield.

A new approach for analyzing oxygenation activity of phagocytes, considering their CL response as a time-probabilistic process, enabled to separate the CL response into two bands and to assign them to the extracellular and intracellular components [Magrisso M. et al.: J. Biolumin. Chemilumin. 10 (1995) 77-84]. Further development of the above approach led to a more accurate analysis of the CL response of phagocytes, providing a three-component resolution of the CL signal corresponding to three different mechanisms of the ROS formation [Magrisso et al.: J. Biochem. Biophys. Methods 30 (1995) 257-69]. The component analysis of CL kinetics further enabled to define kinetics parameters which were correlated to different functional states of phagocytes, such as C-E-V parameters [Magrisso M. et al.: Luminescence 15 (2000) 143-51], however, more detailed information seems to be obtainable by employing other parameters. It is therefore a further object of the invention to provide a method and a device for quantifying the ROS production by phagocytes, utilizing the component analysis of the CL signal.

Other objects and advantages of present invention will appear as description proceeds.

### SUMMARY OF THE INVENTION

The invention provides a method of assessing the in vivo dynamic state of phagocytes in a subject by measuring chemiluminescent (CL) kinetics resulting from reactive oxygen species (ROS) formation in vitro in a biological sample obtained from said subject and containing said phagocytes, said method comprising i) dividing said sample to a plurality of portions; ii) contacting the first portion of said sample with a chemiluminescent substrate, and with a stimulating agent, and measuring a first CL signal, thereby obtaining a first kinetics; iii) exposing the second portion of said sample to an agent or to conditions leading to a partial priming, and contacting said second portion with a chemiluminescent substrate and with a stimulating agent, and then measuring a second CL signal, thereby obtaining a second kinetics; iv) optionally repeating step iii) for the third portion and for all other portions of said plurality of portions obtained by dividing said sample, thereby measuring a third and all other CL signals, constituting a plurality of signals, thereby obtaining a third kinetics and all other kinetics, constituting a plurality of kinetics; v) analyzing said first kinetics, said second kinetics, and optionally said plurality of kinetics, comprising resolving each kinetics into at least three components (subkinetics) having maxima at least at three different times, the components corresponding to at least three different mechanisms of ROS formation; and vi) calculating CL parameters, characterizing the CL kinetics and the subkinetics obtained with and without said priming agent or conditions, and characterizing the relationships between the kinetics. Some mechanisms contributing to the measured CL signals are discussed below, and as already mentioned, the cellular processes underlying the ROS formation and the CL kinetics are rather complex, and the resulting observed signal is compounded of several subsignals. In a preferred embodiment of the invention, three components (subkinetics) having maxima at three different times are considered. The measurements and their processing are performed on biological samples which may belong to unknown patients whose medical state should be clarified, or which may belong to subjects with known clinical states, the latter case enabling to build a database of standard values to be used in assessing unknown states of the subjects, the former case providing data to be compared with the standard values, enabling to evaluate the state of the patient. Preferably, as many relevant conditions, associated with the phagocyte state changes, are included in the broad database to be used; of course, only a part of the data may be utilized, and predetermined relevant conditions may be taken into consideration in case of a specific patient. Generally, said subject exhibiting a certain diagnostic status is selected from the group consisting of a patient to be diagnosed, a healthy subject, a subject suffering from a defined medical condition, a subject undergoing a defined medical treatment, and a subject exposed to defined environmental or other conditions affecting the dynamic state of phagocytes. The method of the invention, thus, preferably comprises creating a database of standard values of said CL parameters, by employing the above said steps i) to vi) on predetermined test groups of subjects, the subjects in each group exhibiting certain known diagnostic status, and by obtaining statistical characteristics of the measurements of each parameter for all subjects in each group, thereby obtaining a standard value of said parameter for said known diagnostic status. Further, the method preferably comprises comparing the CL parameters of said patient to be diagnosed with said standard values. In another aspect, the CL parameters obtained for said patient by the method of the invention may be compared with other reference values than said standard values, said reference values being, for example, published data or values calculated from said published data. Alternatively, said reference values may be the CL parameters characterizing said diagnostic condition, obtained by other means than described above. Said test group of subjects is selected from a group of healthy subjects, a group of subjects suffering from a defined medical condition, a group of subjects exposed to certain environment, and a group of subjects undergoing a defined medical, or other, treatment. Said stimulating agent is preferably selected from the group consisting of optical fiber surface, opsonized zymosan, opsonized synthetic materials capable of fixing complement or eliciting specific antibody expression, opsonized attenuated bacteria, liquid stimulants, and combinations thereof. Said biological sample may comprise a diluted or undiluted biological fluid selected from the group consisting of whole blood, synovial fluid, amniotic fluid, cerebrospinal fluid, peritoneal fluid, pleural fluid, and pericardial fluid. Said phagocytes may be neutrophils, monocytes, eosinophils, dendritic cells, and combinations thereof. The term priming, as used herein, meaning priming of phagocyte respiratory burst, refers to a phenomenon of phagocyte modulation by ligands or conditions, which do not directly stimulate a phagocyte respiratory burst (i.e. do not cause or initiate said burst), but which modulate phagocyte behavior after stimulation (i.e. change the properties of the burst like its intensity, duration, time shape, etc.). Said agent leading to priming, called also a priming agent, may be selected from C5a, C5a.sub.desArg, N-formyl-methionyl peptides, leukotrienes, latelet activating factor, lipopolysaccharide, myeloid colony stimulating factors, cytokines, interferons, interleukins, chemokines, incubation (aging) at predetermined conditions, and combinations thereof. Certain conditions may simulate effects achieved by said priming agents, conditions leading to priming include incubating a sample of phagocytes at predetermined temperature for a period of time, which is called aging. Said priming agent is present under conditions sufficient to shift the current physiological state of the phagocytes, resulting in an enhancement of the phagocytes ability to form the ROS and to elicit the CL reaction. In a preferred embodiment of the method, the priming agent is present under the conditions (relatively low concentration, low temperature and short duration) when the potential of the phagocytes is not affected to give the maximal response (partial priming), the priming being preferably lower than 50% of the priming required for eliciting the maximally enhanced CL signal. In case of fMPL, for example, such a priming concentration may be in the range of 1 to 100 nM, preferably from 5 to 50 nM when applied at 37° C. for 1-5 minutes duration. Of course, each experimental configuration, comprising different types of phagocytes, stimulating agents, priming agents, etc., will have its optimal ranges of reagents, easily determined by a skilled person in accordance with the invention and in order to attain the desired aims. Said chemiluminescent substrate may comprise luminol, isoluminol, or lucigenin. All reagents may be used according to the need, as solids, as solutions, stock solutions, suspensions, attached or bound to surfaces, such as surfaces of reaction chambers, etc. The solvents may comprise non-aqueous solvents provided that their type or amount does not interfere with the CL reaction. The CL light may be monitored by a photometric instrument, comprising a luminometer, a microscope photometer, or a fiber optic sensor. In a preferred embodiment, the instrument comprises optical fibers in direct contact with the phagocyte sample. The mentioned three subkinetics correspond to three different mechanisms of ROS formation, the first of which comprises extracellular process, the second of which comprises an intracellular process, and the third of which comprises a process not directly connected with phagocytosis; additional contributions might be identified, one of which, for example, can be associated with extracellular emission not related to phagocytosis. The related phenomena are explained, for example, in Magrisso et al. [Magrisso M. et al.: Luminescence 15 (2000) 143-151]. The kinetic CL curves depend also on the reaction conditions, such as temperature, reagents concentrations, etc. The first subkinetics may have a maximum, for example at from 1 to 3 minutes at 37°, the second subkinetics usually at from 4 to 7 minutes, and the third subkinetics at more than 7 minutes. The parameters that are used for calculations, and intermediate calculations, intending to characterize the kinetics and subkinetics, may comprise, in various stages of the processing procedures, such values as total CL counts, total CL counts per phagocyte, counts per the whole kinetics or its subkinetics, the times corresponding to the maxima on kinetic curves, areas under kinetic curves, ratios providing normalized values, background CL counts, combinations of the values such as Capacity (C), Effectiveness (E), and Velocity (V), or derivatives of some of the parameters, etc. Said derivative may, generally, comprise a recalculated value, or, specifically, it may comprise a small change of one parameter resulting from a small change in another parameter. Said normalization is a correction of the recorded signal to predetermined number of phagocytes, and/or to the erythrocyte interference. When speaking about CL kinetics, as a skilled person understands, the time dependence of CL signal is meant, and, sometimes, in certain contexts, kinetic curves may be intended. The parameters may relate to a stimulated sample, to a sample primed under certain priming condition or with certain priming agent, to an aged sample, to a sample of the patient whose clinical state is assessed, to a control sample, or to their combinations. Said analyzing, according to the method of the invention, comprises determining the contributions of intracellular and extracellular ROS forming processes, preferably utilizing the resolution into three components, utilizing, e.g., a technique as described in Magrisso et al. [Magrisso M. et al.: Luminescence 15 (2000) 143-151], wherein said components correspond to time-probabilistic curve associated with statistically significant mechanism leading to the production of CL by phagocyte. Any means, known in the art, for assessing significance of measured or calculated parameters, or any procedures for analyzing data, or for distinguishing contributing sub-bands in the measured signals, may be utilized when processing data in the method of this invention. Said procedures may comprise, for example, multiple discriminant analysis, the least square minimization, nonparametric statistics, etc. The dynamic state of patient's phagocytes may reflect various disorders, and the parameters reflecting dynamic state may be correlated with said disorders. Therefore, important diagnostic information can be obtained by comparing said parameters reflecting the instantaneous patient's dynamic state with standard values of such parameters obtained by analyzing large groups of patients belonging to certain diagnostic group. Said standard values for a group of subjects exhibiting certain diagnostic condition are obtained by measuring chemiluminescent (CL) kinetics involved in the ROS formation in vitro in biological samples obtained from said subjects, the method comprising i) dividing said sample to a plurality of portions; ii) contacting the first portion of said sample with a chemiluminescent substrate, and with a stimulating agent, and measuring a first CL signal, thereby obtaining a first kinetics; iii) exposing the second portion of said sample to an agent or to conditions leading to a partial priming, and contacting said second portion with a chemiluminescent substrate and with a stimulating agent, and then measuring a second CL signal, thereby obtaining a second kinetics; iv) optionally repeating step iii) for the third portion and for all other portions of said plurality of portions obtained by dividing said sample, thereby measuring a third and all other CL signals, constituting a plurality of signals, thereby obtaining a third kinetics and all other kinetics, constituting a plurality of kinetics; v) analyzing said first kinetics, said second kinetics, and optionally said plurality of kinetics, comprising resolving each kinetics into three components having maxima at least at three different times (three subkinetics), the components corresponding to at least three different mechanisms of ROS formation; vi) calculating predetermined independent CL parameters characterizing the kinetics and subkinetics obtained with and without said priming agent, thereby obtaining a first measurement of said standard value for each independent CL parameter; and vii) repeating steps i) to vi) for samples obtained from a second, third, and all other subjects in said group of subjects exhibiting said diagnostic condition, thereby obtaining a second, third, and other measurements of said standard value; and viii) calculating from said first, second, third, and all other measurements obtained in steps v) and vii), the mean value and the desired statistical factors for each independent CL parameter, thereby obtaining the required standard value of said CL parameter for said diagnostic condition. Any magnitudes necessary for evaluating the significance of the results, their reliability, and characterizing the distribution of results and their other features, whether clarifying the statistical or diagnostic aspects, are calculated by methods known in the art. Said predetermined independent parameters are selected so as to differentiate best, in a statistically significant manner, between two or more groups of subjects exhibiting different diagnostic conditions. Each diagnostic condition will provide different set of standard CL parameters. Said diagnostic condition may be any medical condition or disorder. Some effects of various disorders on phagocytes are known, and others may be disclosed by means of the present invention. The suspected conditions may comprise infection, inflammation, and immunity disorder. Various conditions to be considered in the context of the invention may comprise, for example, peritonitis, tunnel infection, diabetes, suppression after transplantation, bacterial infection or other antimicrobial infection, and viral infection. Thus, the method of the invention comprises assessing the in vivo functional state of phagocytes in a human or animal patient by determining the normalized amounts and proportions of extracellularly and intracellularly generated ROS during interactions of said phagocytes contained in a biological sample with a stimulating agent, comprising i) determining the approximate number of phagocytes and erythrocytes in said sample; ii) determining the extents of extracellularly and intracellularly phagocytes-generated ROS over a predetermined time period in a first portion of said sample; iii) determining the extents of extracellularly and intracellularly phagocytes-generated ROS over said time period in a second, and optionally in a third portion and in other portions of said sample, which second portion and other portions were exposed to an agent or conditions causing a partial priming which shifted the functional state of the phagocytes in said samples, wherein said priming agents and conditions are different in all portions; iv) comparing the extents and their proportions of said first portion, with the extents and their proportions of said second portion, and optionally also of said third and other portions, of the sample, obtaining parameters reflecting said functional state of phagocytes; and v) comparing said parameters obtained in step iv) with a range of controls, enabling to assess the functional state of the phagocytes.

The invention further provides a method for testing an effect of a pharmacologically important agent on phagocytes by analyzing in vitro interactions of the phagocytes and the agents. The method comprises measuring chemiluminescent (CL) kinetics resulting from reactive oxygen species (ROS) formation in vitro in a biological sample containing phagocytes, which measuring comprises i) dividing said sample to a plurality of portions; ii) contacting the first portion of said sample with a chemiluminescent substrate, and with a stimulating agent, and measuring a first CL signal, thereby obtaining a first kinetics; iii) exposing the second portion of said sample to conditions leading to a partial priming or to an agent leading to a partial priming, and contacting said second portion with a chemiluminescent substrate and with a stimulating agent, and then measuring a second CL signal, thereby obtaining a second kinetics; wherein said stimulating agent in steps ii) and iii) and said agent leading to a partial priming (priming agents) are either standard agents or tested agents; iv) optionally repeating step iii) for the third portion and for all other portions of said plurality of portions obtained by dividing said sample, thereby measuring a third and all other CL signals, constituting a plurality of signals, thereby obtaining a third kinetics and all other kinetics, constituting a plurality of kinetics; v) analyzing said first kinetics, said second kinetics, and optionally said plurality of kinetics, comprising resolving each kinetics into three (or more) components having maxima at three different times (subkinetics), the components corresponding to at least three different mechanisms of ROS formation; vi) calculating CL parameters, characterizing the kinetics and the subkinetics obtained with and without said priming agent, and characterizing the relationships between the kinetics; and vii) comparing the CL parameters obtained in steps i) to vi) for standard agents with the CL parameters obtained in the same steps for tested agents; wherein standard agents are any agents whose effect on the phagocytes is known, and the tested agents are agents whose effect of the phagocytes is examined. In a preferred embodiment, the method of testing an effect of a pharmacologically important agent on phagocytes preferably comprises i) providing a biological sample containing phagocytes, and determining the approximate number of phagocytes in the sample; ii) contacting a first portion of said biological sample with a stimulating agent, optionally after contacting a priming agent, and with a chemiluminescent substrate, and measuring a first CL kinetics; iii) determining the amounts of extracellularly and intracellularly phagocytes-generated ROS over a predetermined time period in said first portion; iv) contacting a second portion of said sample with said tested agent to shift the functional state of the phagocytes, and then contacting said sample with the stimulating agent and with the chemiluminescent substrate, and measuring a second CL kinetics; v) determining the amounts of extracellularly and intracellularly phagocytes-generated ROS over a predetermined time period in said second portion; vi) comparing the amounts and proportions of extracellularly and intracellularly phagocyte-generated ROS of the first and the second portions of the sample, thereby obtaining the in vitro effect of the tested agent on the phagocytes; optionally vii) comparing said second CL kinetics with CL kinetics corresponding to a range of control phagocytes-samples obtained from patients exhibiting a range of diagnostic conditions, thereby comparing the effect of said tested agent with an effect of various diagnostic conditions on the phagocytes, and optionally viii) comparing said second CL kinetics with CL kinetics corresponding to a range of control phagocytes-samples treated according to steps i) to vi), wherein instead of said tested agent one or a plurality of other pharmacologically important agents were used, whose effect on the phagocytes is known, thereby comparing the effect of said tested agent with an effect of various other pharmacologically important agents. Clearly, every standard component of the reaction system can become a tested component (stimulating agent, priming agent, CL substance, phagocytes, serum), if other components are kept constant.

The invention is directed to an apparatus for determining the in vivo dynamic state of phagocytes in a subject, comprising i) at least one sensor for measuring a CL kinetics in a biological sample containing phagocytes in contact with a stimulating agent, and optionally with a priming agent, and with a CL substrate; and ii) a processor for resolving said CL kinetics into three subkinetics corresponding to three different mechanisms of ROS formation. The apparatus of the invention preferably measures simultaneously or consequently at least two CL kinetics in at least two portions of one sample, wherein the two portions differ in the concentrations of said stimulating and/or priming agents. Said apparatus i) obtains from said sensor a signal corresponding to two different kinetics, and resolves each of the kinetics into three subkinetics; ii) calculates CL parameters characterizing the kinetics and subkinetics and their relation; iii) and compares said CL parameters with standard values of said parameters, stored in the memory, corresponding to a range diagnostic condition; and iv) provides an assessment of the in vivo dynamic state of the patient's phagocytes. Said subkinetics are preferably approximated by Poisson distribution curves. In a preferred embodiment of the invention, the apparatus for determining the it vivo dynamic state of phagocytes in a subject comprises an optical fiber that is in direct contact with said sample containing phagocytes. The apparatus preferably measures simultaneously a plurality of portions divided from one sample, and/or a plurality of samples obtained from plurality of subjects. In a preferred embodiment of the invention, the apparatus for determining a functional state of phagocytes of a subject comprises a sensor for single or multiple measurements of CL kinetics involved in generating ROS over a predetermined time period in a phagocyte-containing biological sample of a patient; and a processor for determining the extent of extracellularly and intracellularly generated ROS.

In an important aspect of the invention, a method is provided for measuring CL kinetics resulting from ROS formation in vitro in a patient's sample containing phagocytes in direct contact with an optical fiber.

The invention is also directed to a kit for use in the evaluation of the in vivo dynamic state of phagocytes, of a patient, comprising i) disposable chamber(s) for measuring CL kinetics, or parts of the chamber in which said measuring occurs, involved in the ROS formation in a biological sample containing the phagocytes obtained from said patient; ii) an opsonized, oxidative metabolism stimulating, agent; iii) a chemiluminigenic substrate; and iv) a priming agent in an amount sufficient to obtain phagocytes with a shifted functional state in a portion of said sample, but in an amount lower than amounts eliciting maximal response.

This invention, thus, provides a method of assessing the dynamic change of the phagocyte functional status. The method is suitable for assessing the momentary state of the circulating phagocytes in vivo by perturbating their reaction in vitro. The method involves making at least two consecutive tests of the same biological sample. The current invention shows that the understanding of problems associated with pathological processes can be improved by methods for monitoring and assessment of functional status of circulating phagocytes by the amount of extracellular part and intracellular part of phagocyte generated reactive oxygen species (ROS). The assessment is performed after stimulation of phagocyte respiratory burst using data obtained from the same sample, which permits to quantify properly the relative contribution of intracellularly and extracellularly generated ROS to the total oxidative phagocyte response. The functional state of phagocytes is determined by performing the following steps: (i) contacting a first portion of a phagocyte containing biological sample from the patient with a stimulating respiratory burst agent (e.g., optical fiber surface, opsonized zymosan, opsonized synthetic materials capable of fixing complement or eliciting specific antibody expression, opsonized attenuated bacteria and combinations thereof) and measuring the response with a chemiluminescent substrate; (ii) contacting a second portion of the biological sample from the patient with a phagocyte priming agent (e.g., C5a, C5a.sub.desArg, N-formyl-methionyl peptides, leukotrienes, latelet activating factor, lipopolysaccharide, myeloid colony stimulating factors, incubation at a predetermined temperature, and combinations thereof, a stimulating respiratory burst agent, and measuring the response with a chemiluminescent substrate; and (iii) comparing the relative contribution of extracellularly and intracellularly produced reactive oxygen products by their chemiluminescent response of the first and second portions of the sample as a measure of the patient's dynamic phagocyte functional status. Thus, the method of assessing the in vivo dynamic state of phagocytes in a patient according to a preferred embodiment of the invention divides a biological sample, obtained from a subject to be checked, to a plurality of portions; measures a plurality of CL kinetics in vitro—the first one without priming and the second, third, and others, with different priming agents or conditions; resolves each kinetics into at least three components having maxima at, at least, three different times; calculates predetermined independent CL parameters; and compares said CL parameters with standard values contained in the database which is continually broadened by new measurements; thereby assessing the state of phagocytes, and possibly also assessing diagnostic state of the subject reflected by the phagocytes state. As for the number of parameters processed in calculations, if a subjects sample is divided, for example, to four portions intended for three different types of priming, and if for each of the four obtained kinetics all 16 parameters of Table 1 are taken into consideration, 64 new parameters are obtained for each subject, and these 64 parameters obtained from many subjects differing by clinical states may be subjected to multiple discriminant analysis. The number of parameters included in the concrete calculation may depend on the diagnostic state that is suspected, on the global diagnostic strategy, etc.

In another aspect, the invention provides a method for analyzing in real time in vitro interactions between phagocytes and an agent to be tested, by determining the normalized amounts and proportions of extracellularly and intracellularly generated ROS, during interactions of phagocytes contained in a biological sample with said agent. A first portion of a phagocyte containing biological sample is contacted with an agent stimulating respiratory burst and with a chemiluminescent substrate, providing a first measurement. A second portion of the biological sample is then contacted with said tested agent, potentially stimulating the respiratory burst, and a chemiluminescent substrate. Finally, the relative contributions of extracellularly and intracellularly produced reactive oxygen products are compared, by their chemiluminescent responses, for the first and second portions of the sample, as a measure of the in vitro interactions between phagocytes and agent to test.

The apparatus of the invention for determining the dynamic functional state of phagocytes in a patient allows better assessment of the relative contribution of intracellularly and extracellularly generated ROS to the total oxidative phagocyte response. The apparatus comprises a disposable part that significantly facilitates the procedure of performing the tests by providing standard environments for phagocyte activation and decreasing the efforts required for maintenance of the apparatus.

It may be concluded that the method of the invention, together with the apparatus or the invention, provide a novel tool for assessing the condition of a patient with maximal simplicity and minimal invasiveness, wherein the measurement may be repeated with one small sample many times, possibly including various modifications during consequent measurements, with each modification providing still more, diagnostically relevant information. FIG. 5 is illustrative in showing one aspect of the potential of said new tool.

### BRIEF DESCRIPTION OF THE DRAWINGS

The above and other characteristics and advantages of the invention will be more readily apparent through the following examples, and with reference to the appended drawings, wherein:

FIG. 1. shows a model CL kinetics; it shows a graphic representation of chemiluminescent response, its components and their relationship with extracellularly and intracellularly produced reactive oxygen species during phagocytosis; FIG. 1A shows component separation, and their contribution to the total effect, in accordance with one aspect of the invention; FIG. 1B shows CL kinetics and its parts (see legend) directly connected with phagocytosis (sum of first and second component), as well as not directly related to phagocytosis (third component);

FIG. 2. shows the effect of phagocyte priming on the CL kinetics; diluted whole blood samples in vitro were preincubated at 37° C. for 5 minutes in the absence or presence (see the legend) of 3 nmol/liter of N-formylmethionyl leucyl phenylalanine (fMLP); FIG. 2A shows the CL response, and FIG. 2B shows derived kinetic parameters which reflect relative contribution of extracellularly and intracellularly produced ROS (RU=relative units), under the current conditions the effect of fMLP is more significant for the extracellular ROS production;

FIG. 3. shows the effect of glucose on phagocyte activity, the effect on phagocyte priming with D-glucose on the CL kinetics is studied; diluted whole blood samples in vitro were preincubated at 37° C. for 5 minutes in the absence (−) or presence (+) of 5.56 mmol/liter of D-glucose during the incubation (Gi) or during the measurement (Gp); FIG. 3A shows the CL response, and FIG. 3B shows a derived kinetic parameter, capacity, which reflects the sum of ROS related to phagocytosis (extracellularly and intracellularly produced, components 1 and 2) and not-related to phagocytosis (component 3);

FIG. 4. shows the effect of aging of blood on the CL kinetics of phagocytes; the phagocytes were aged in vitro over a five-hour period prior to the measurement; the relative contribution of phagocyte extracellularly and intracellularly produced ROS was measured; FIG. 4A shows CL response, FIG. 4B shows derived kinetic parameters, effectiveness and velocity; the phagocyte functional status undergoes continuous transition from “resting” state (high efficiency, low velocity) to “stand-by” state (decreased efficiency, higher velocity);

FIG. 5. is a graphic representation of chemiluminescence response of the phagocytes, in accordance with the invention, of a patient during the course of the treatment showing the effect of healing on the relative contribution of phagocyte extracellularly and intracellularly produced ROS; data were acquired through repetitive testing of the patient during an 18-days period of successful treatment of pulmonary abscess;

FIG. 6. shows the effect of pharmacological products, either immunomodulators or allergens, on CL kinetics of phagocytes; diluted whole blood samples in vitro were preincubated at 37° C. for 5 minutes in the absence or presence (see the legend) of the agent; FIG. 6A shows the CL response for treatment with 1.5 mmol/liter of aspirin, and FIG. 6B shows the CL response for treatment with 1 U or 5 U of IgE; aspirin caused a significant decrease of phagocytosis-related parts of the CL response, IgE caused a drastic decrease of the velocity of respiratory burst;

FIG. 7. shows the effect of industrial pollutants, such as metals, on phagocyte activity; diluted whole blood samples in vitro were preincubated at 37° C. for 5 minutes in the absence or presence (see the legend) of the metals (Fe3+, Cu2+);

FIG. 8. shows the effect of temperature on the CL response; diluted whole blood samples in vitro were preincubated at 20 or 37° C. for 5 minutes in the presence of zymosan particles, the phagocytes were stimulated by a fiber surface; silica material of optical fiber stimulates phagocytes to produce “frustrated phagocytosis” leading to clear indication of the extracellularly produced light and its time appearance (see the arrows);

FIG. 9. shows the effect of zymosan quantity (0.5 or 4.0 mg/ml) on the stimulation of the phagocytes; the effect of tuned phagocytosis on the relative contribution of extracellularly and intracellularly produced ROS is illustrated;

FIG. 10. shows schematically the luminometer according the invention; FIG. 10A is a block diagram, in which the numbers have the following meanings: 1—thermo-controlled fiber holder; 2—sample-fibers; 3—photon-counting Photomultiplier Tube (PMT) detector; 4—power supply; 5—Programmable Logic Controller (PLC); 6—interface; 7—computer; 8—step motor; 9—position sensor; 10—thermo controller; 11—rotating disk-shutter; FIG. 10B is a sectional view of the upper thermo-regulated part of the fiber holder, the arrows point to the cuvette and fiber positions;

FIG. 11. outlines the momentary and dynamic innate immune system status; CL1 and CL2 are two different parameters of respiratory burst, obtained from CL kinetics; the points denoted as S1, S1′, S0, S2, S2′ depict different momentary innate immune statuses, wherein ΔCL1 and ΔCL2 show dynamic changes in case of two different scenarios;

FIG. 12. shows representative kinetics data of follow-up dialysis patients (FUP), recorded and derived for the whole blood samples comprising “standard system” (S), “primed system” (P), and “aging system” (A); FIG. 12A shows the kinetic parameters for the three systems (standard, primed, and aging) calculated according to the invention; FIG. 12B shows the CL response curves; FIG. 12C shows resolving into three components as explained in FIG. 1A;

FIG. 13. shows a correlation map of diagnostic cases in the two-dimensional space, obtained by discriminant analysis; calculated parameter CL2 is plotted against calculated parameter CL1 for a group of patients exhibiting various diagnostic states; each case is shown by a small symbol, large symbol depicting the mean canonical group coordinates; included are follow-up cases (FUP), tunnel infection (TINF), peritonitis (PER), patient suppressed after transplantation (SUPR), diabetes mellitus (DIAB), and cases during treatment (TRANS);

FIG. 14. shows the comparison of PER, FUP, and CTR groups of cases; FIG. 14A shoes separation by discriminant analysis using multi-parameter linear functions to form the axis CL1 and CL2; FIG. 14B shows the mean values, ±SE values, and ±SD values for ReExtra SP values; FIG. 14C shows the mean values, ±SE values, and ±SD values for velocity SP values; and

FIG. 15. represents case separation in accordance with the invention of four pre-determined groups of subjects, comprising CAPD (continuous ambulatory peritoneal dialysis) cases; FIG. 15A is based on processing 15 CL parameters obtained from measuring stimulated portion and a fMLP-partially primed portion; the patients belong to the following: follow-up, peritonitis, diabetes, and healthy cases; FIG. 15B is based on 11 CL parameters obtained form measuring stimulated portion and an aged-primed portion; CL parameters determined from two measurements; the patients belong to the following: peritonitis, suppresses, diabetes, and healthy cases.

### DETAILED DESCRIPTION OF THE INVENTION

It has now been found that more information may be obtained about the in vivo state of phagocytes, when the CL response measurements of phagocyte-generated ROS are carried out after shifting the functional state of said phagocytes, while resolving the separate contributions of extracellularly and intracellularly generated ROS. Particularly, in vitro measurements resolving the extracellular and intracellular contributions provide unexpectedly better results, when said shifting is a partial shifting, for example achieved by a partial priming of the phagocytes in vitro. When analyzing CL responses in phagocyte samples obtained from different subjects and calculating kinetics parameters, it has been observed that said parameters have remarkable ability to distinguish between different diagnostic states of said different subjects. Moreover, it has been found that additional information may be obtained when measuring a plurality of portions of one sample, in which portions different priming conditions were employed.

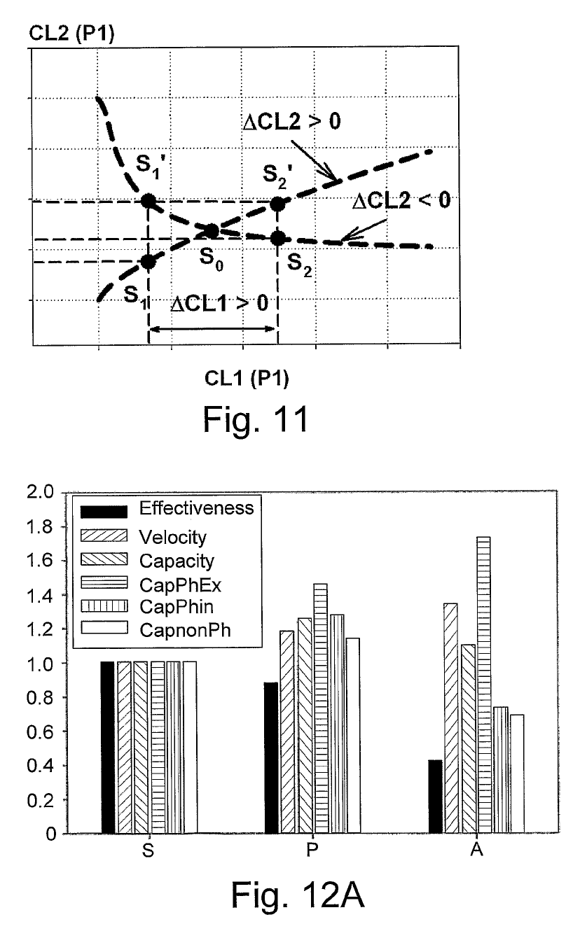

In a preferred embodiment of the method according to this invention, the in vivo dynamic state of phagocytes is assessed by measuring ROS generated in vitro in a sample containing said phagocytes during the interaction of said phagocytes with a stimulating agent and a chemiluminescent substrate, said agent being an opsonized factor naturally inducing phagocyte response in vivo or a factor simulating same, and said substrate being a material emitting CL light in the presence of ROS, wherein the obtained measurement, in the form of CL signal—time curve (response curve), is processed to resolve the extracellular and intracellular contributions using, for example, analysis as described in Magrisso et al. [Magrisso M. et al.: Luminescence 15 (2000) 143-51]. Said analysis may provide a set of parameters (CL parameters) that enable good separation and characterization of the two contributions. The above measurement is performed at least twice with the same sample containing said phagocytes, with two different amounts of a priming agent. In a preferred embodiment of the invention, one measurement is carried out in the absence of a priming agent, and the other in the presence of a priming agent, optionally using more types of priming conditions providing a plurality of primed CL signals, which priming agent affects the dynamic state of the phagocytes, said priming agent may be, for example, fMLP. Said two, or more, measurements comprising said processing enable to extract maximum information from the CL signals, and provide a sensitive tool for diagnostically distinguishing between samples containing phagocytes in different dynamic states. In the method of the invention, the two measurements are carried out in at least two portions of a biological sample, wherein a shift in the dynamic state caused by the priming agent is preferably small, so that the change in the dynamic state comprises rather a perturbation than reaching the maximal modulation potential. Generally speaking, the measurements should provide for said CL parameters a tendency of their change by using partial priming rather than maximal priming (see, e.g., FIG. 11). Various parameters, obtained by the measurements, may be plotted in multidimensional arrangements against each other. For example, the following procedure may provide a two-dimensional space of diagnostic conditions: i) plotting the standard value (whose acquiring represents one aspect of the invention) of a first CL parameter against the standard value of a second CL parameter for a first diagnostic condition (e.g., two standard parameters for “infection” are plotted: CL_{inf 1} against CL_{inf 2}); ii) repeating step i) for a second, a third, and other diagnostic conditions (e.g., the same types of standard parameters for “diabetes” are plotted: CL_{diabet 1} against CL_{diabet 2}, etc.), thereby obtaining a two-dimensional graph in said space of diagnostic conditions; iii) plotting said first CL parameter found in an examined patient against said second CL parameter found in the same patient; and finally iv) assessing the state of the patient according to the position of his/her point in the space of diagnostic conditions. If the patient's position is closer in the diagnostic space to the area of a certain disorder, it may indicate that such disorder might be suspected, and corresponding known tests should be preferably performed. Said two-dimensional space is alternatively used for placing CL₁-CL₂ points corresponding to a plurality of patients exhibiting certain medical condition, and the dispersion of the points among patients of one type is thus visualized. More than two parameters will create a multidimensional space of conditions (see, e.g., C-E-V space described below).

The invention provides a sensitive, specific and rapid diagnostic method and device, which enable to timely obtain clinically relevant diagnostic and management information for patients undergoing an infection. The change of phagocyte functional status is indicative of an infection. The invention quantifies the phagocyte functional status using the CL pattern resulting from generated ROS. When processing CL signals in the method of the invention, the following factors may be considered, as partly revised in Magrisso et al. [Magrisso M. et al.: J. Biolumin. Chemilumin. 10 (1995) 77-84]. A typical sample containing 10⁴-10⁵ cells may provide about 10⁵ counts within a 30 min interval, making approximately 1 count per cell during this whole time interval. So the events observed are very rare. Therefore, a Poisson-type distribution has been employed here, which describes processes whose probability of occurrence is small. A component of the chemiluminescent kinetics is formed after PMNs stimulation, wherein the CL intensity rises from the background value through a maximum, and returning to the background again during the time of measurement. As shown previously [Magrisso M. Ibid], a Poisson-type distribution is suitable for describing the shape of the instant CL signal, as well as for sub-components to which the CL signal is resolved, as follows:

$${I}_{i} ( t ) = {N}_{i} {λ}_{i} {m}_{i} \frac{{( {λ}_{i} t )}^{{m}_{i}}}{{m}_{i} !} {e}^{- {λ}_{i} t} \qquad ( 1 )$$

Wherein I is light intensity, t=time, λ=the average number of registered photons per time unit, m=the capacity of a luminous centre to emit photons, and N=the number of centers of the same registered type (with same λ and m), the values relate to the i-th component. N depends on the size of the stimulated areas upon the cell surface and on the concentration of the luminol used. General concepts of the model approach may comprise the following CL parameters:

Chemiluminescent capacity of one component (S)—This is the whole quantity of light emitted during the response of the component; Si=Ni×mi (where i=1, 2, or 3 is the number of the component). It is equal to the area under its chemiluminescent kinetics.

Chemiluminescent capacity of the whole response (C)— This is the sum ΣNi×mi, which is equal to the area under the whole CL kinetics. The real CL kinetic data are modeled on the basis of Equation (1). The values of the component parameters are calculated using an iteration procedure to obtain the minimum sum of the squared differences between the real and the model CL intensity. Each component contributes to the total intensity, depending on its own kinetics. It must be pointed out that this is possible no matter whether or not different phases are visible in the total kinetics. Using the component-model terms, the different functional states of neutrophils can be characterized by capacity, effectiveness and velocity of the respiratory burst occurring after a stimulation [Magrisso M. et al.: Luminescence 15 (2000) 143-51]. These parameters can be defined as follows:

Capacity (C)—The total CL capacity, as defined above, of predetermined number of cells, which reflects their capability to generate ROS.

Effectiveness (E)—The ratio of the capacity of the second component to that of the first. As mentioned above, the capacities of the first and second components are closely connected with extracellular and intracellular ROS generation during phagocytosis, respectively. Hence, the above ratio shows the effectiveness of ROS generated during phagocytosis.

Velocity (V)—The ratio of the sum of the capacities of the first and second components to the capacity of the third component of CL kinetics, with its increasing values, the respiratory burst is achieved faster [Magrisso, Ibid.}.

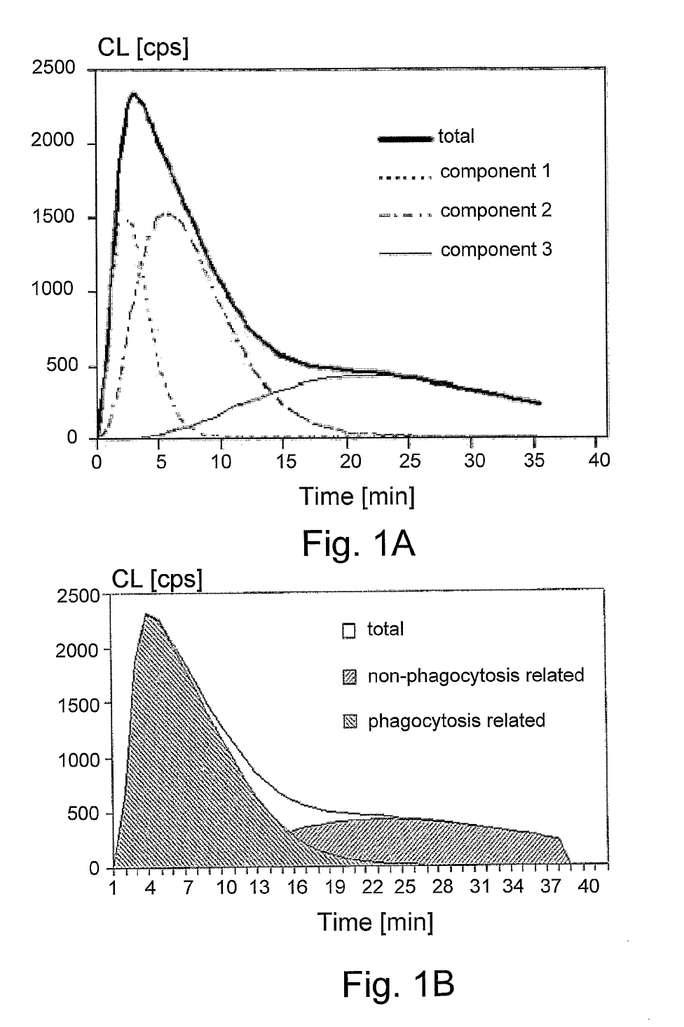

When processing the measurements in the method of the invention, each recorded kinetic CL curve is presented as a sum of at least three components, as explained in Magrisso et al. [Magrisso M. et al., J Biochem. Biophys. Methods 30 (1995) 257-69]. The time dependence of CL intensity, recorded after zymosan stimulation of PMNs, is exemplified in FIG. 1. The model components of the total CL kinetics are shown, wherein the cellular-biochemical characteristics of the three components are summarized as follows [Magrisso et al., Ibid]:
The first component represents processes that take place near the plasma membrane. They are connected with phagocytosis and cause extracellular CL.
The second component represents processes located inside the cell. They are connected with phagocytosis and cause intracellular CL.
The third component mainly represents processes that lead to intracellular CL. However, they are not directly connected with phagocytosis (see FIG. 1B).

Using the above parameters C-E-V as coordinates of three-dimensional space (CEV space), a particular state of PMNs can be visualized in that space relatively to the other states. Each point of this space corresponds to different functional potential of PMNs for ROS generation. A part of this space is considered as a normal, for example “resting”, which has low Capacity, low Velocity and high Effectiveness. Other CEV space areas are characteristic for various known medical conditions. Different momentary functional states of phagocytes were considered in Magrisso et al. [Luminescence 15 (2000) 143-51], based on the calculated parameters of the three components, said phagocyte states were classified, wherein the states are associated with the phagocyte status in regard to the respiratory burst, the classification including the following states: “resting”, “stand-by”, “fighting”, “effective”, “restoring”, “frustrated”, “alternatively-activated”, and “frustratedly-activated”. Phagocyte functional state in the blood refers to the readiness of the circulating phagocyte to produce ROS after a stimulation.

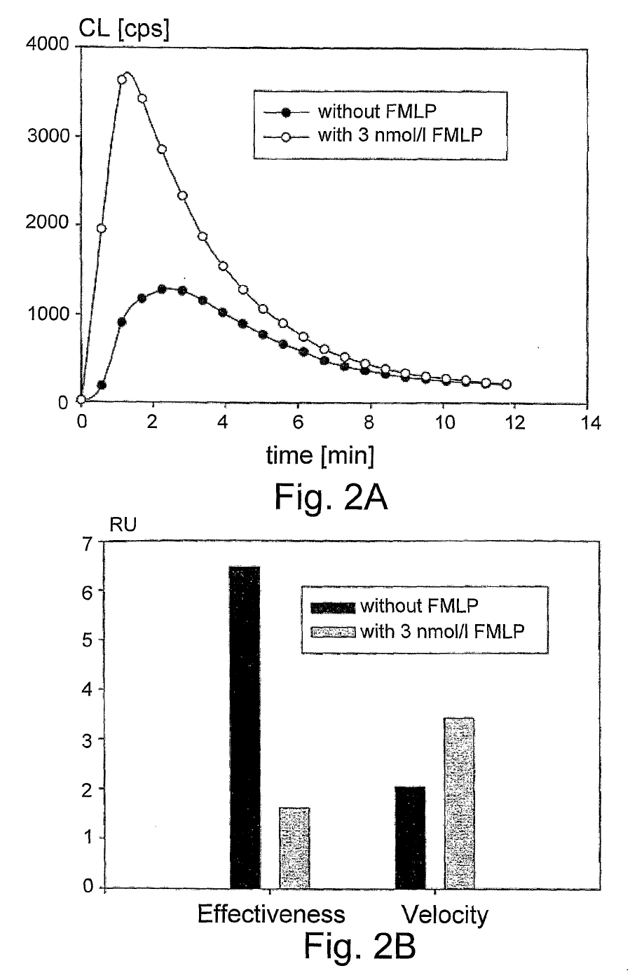

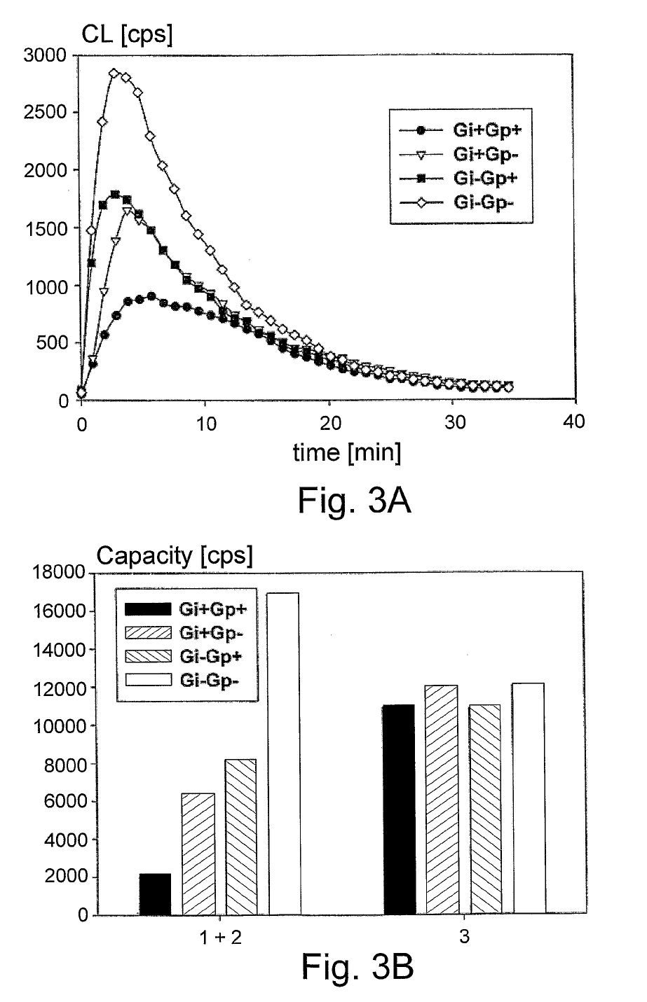

In one aspect of the invention, dynamic functional state of phagocytes is assessed after the stimulation of phagocytes, using data obtained from the same sample without priming and with one or more types of priming, resolving the relative contribution of intracellularly and extracellularly generated ROS to the total oxidative phagocyte response. Firstly, a first portion of a phagocyte-containing biological sample is contacted with an agent stimulating the respiratory burst and with a chemiluminescent substrate. Secondly, a second portion of a phagocyte-containing biological sample is contacted with a priming agent, i.e. an agent modulating eventual response of the phagocytes after stimulation, with an agent stimulating the respiratory burst, the burst being visualized as a CL signal in the presence of a chemiluminescent substrate (see FIG. 2 and FIG. 3). The relative contributions of extracellularly and intracellularly produced reactive oxygen products are assessed, serving for the assessment of the dynamic functional status of the patient's phagocytes.

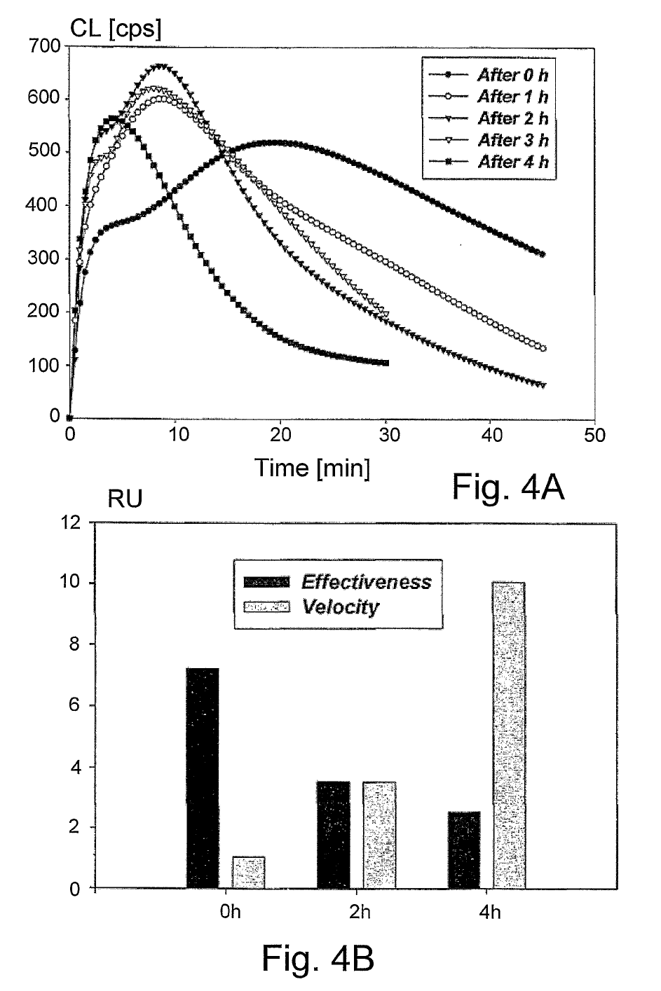

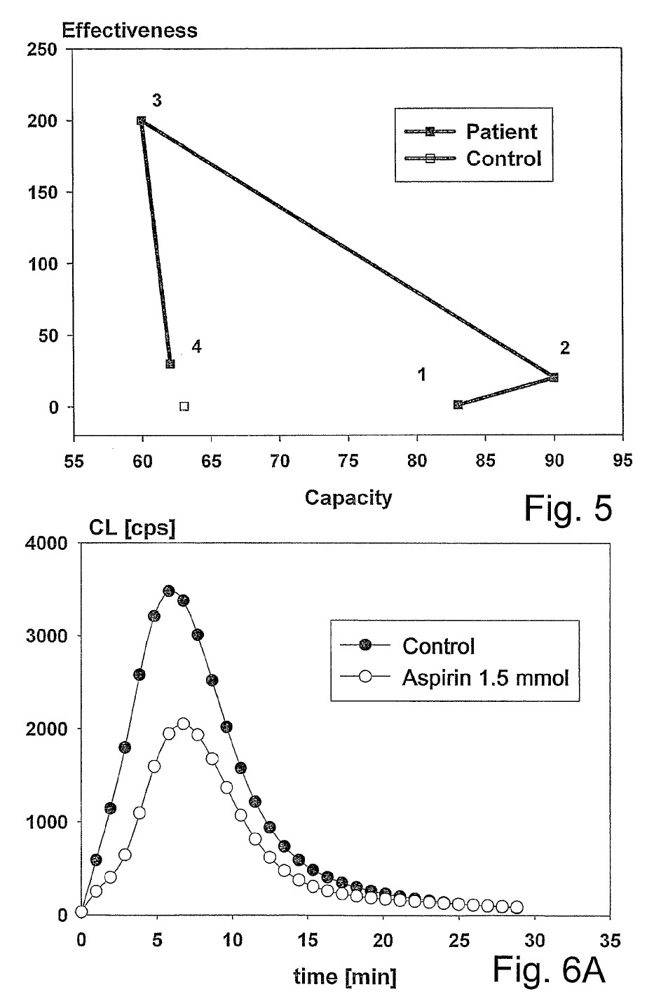

U.S. Pat. No. 5,108,899 characterizes inflammation of a patient by comparing the extent of opsonin receptor expression on phagocytes at certain clinical state in vivo, with the maximum opsonin receptor expression, inducible in vitro. The theory is that the less opsonin receptor expression may be induced, the greater the inflammation. The method of said patent primes and stimulates opsonin receptor expression to give a maximum amount of chemiluminescence with zymosan, without assessing the relative contribution of intracellularly and extracellularly generated ROS. In contrast, the present invention, using a component model of phagocyte emission after stimulation, and providing a plurality of measurements from one sample, enables to extract more information (see, e.g., FIG. 1B, FIG. 4 and FIG. 5).

Whereas the prior technique overstimulates the phagocytes, overloads them with a priming agent, and smoothes out potentially useful information, the invention subjects the phagocytes only to a partial priming. Furthermore, dividing a patient's sample to a plurality of portions, and priming the portions by different agents or conditions, additional characteristics are obtained, enabling more reliably to correlate the measured kinetics with the clinical states of the patients. The experimentally in vitro obtained parameters may reflect numerous clinically relevant states in the subjects, comprising untypical states, pathologic conditions, stages in treatments, presence of drugs, and others. The kinetic measurements according to the invention provide a plurality of parameters, and statistical importance of any of the parameters or of any combinations thereof is easily evaluated and computed by known methods, such as multiple discriminant analysis, so that finally only such quantities that are well correlated with the relevant clinical states, and which form a set of independent parameters, may be selected for further work and uses as predetermined independent parameters. Some groups of subjects, being in a clinically relevant situation, well characterized by other independent known diagnostic methods, will be characterized also by means of said predetermined independent parameters, and the results will be used for creating a database of standard parameters to which the measurements, obtained from subjects with unknown anamnesis or with an unclear diagnostic status, will be compared. Of course, any measurements, even obtained from “unknown” patients, may be used for broadening the database, after confirming the diagnosis with other independent methods. The invention thus, in one aspect, comprises a valuable diagnostic method or auxiliary diagnostic method, that works with ever growing database. The accumulated data will offer further means for optimizing the diagnostic strategy. For example, knowing that the patient is diabetic will affect the selection of parameters to be evaluated, and may reduce the number of measurements.

The method of this invention enables to extract maximal, diagnostically relevant, information about the in vivo state of phagocytes in a plurality of measurements performed on one sample divided into a plurality of portions. In specific situations, one measurement may provide the desired information.

The computing activities may be integrated with a device according to the invention, or may be performed separately, using methods known in the art. As for said multiple discriminant analysis, it is a known statistical technique, but its results depend on the parameter selection to be processed. For example, U.S. Pat. No. 5,108,899 processes parameters simply derived from direct measurements of CL signals with and without full priming, including also white blood count (WBC) as one of the parameters used in the discrimination analysis. This invention, in contrast, includes parameters calculated and derived from a model analysis, enabling to address the momentary state of the phagocytes. In this invention, WBC is a parameter used merely for the normalization. If said biological sample containing phagocytes is blood, a correction of the CL signal is effected in the method of the invention for the phagocyte number (linear correlation), and the erythrocyte number (nonlinear correlation).

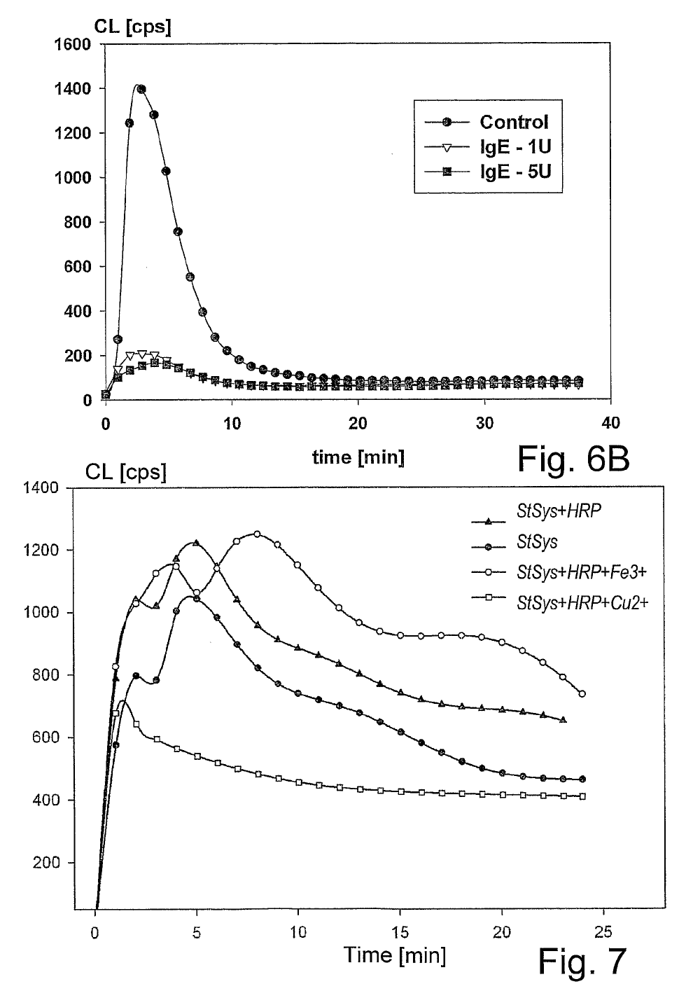

Other aspect of the invention is a method for analyzing in vitro interactions between phagocytes and an agent of potential pharmacological importance by measuring the CL response, while incorporating said tested agent to one portion of the phagocyte sample, before or together or instead of stimulating and/or priming agent, in a method according to the invention as described above, for example, by contacting a first portion of a phagocyte containing biological sample with a stimulating respiratory burst agent and with a chemiluminescent substrate, and then by contacting a second portion of the sample with said agent to be tested together with a stimulating respiratory burst agent and a chemiluminescent substrate, followed by comparing the relative contributions of extracellularly and intracellularly produced reactive oxygen products in the two measurements, to characterize the in vitro interaction between the phagocytes and the agent to test. Such an agent to test may belong, for example, to pharmacological products showing immunomodulating activity (FIG. 6A) or to allergens (FIG. 6B), or to industrial pollutants (FIG. 7), etc.

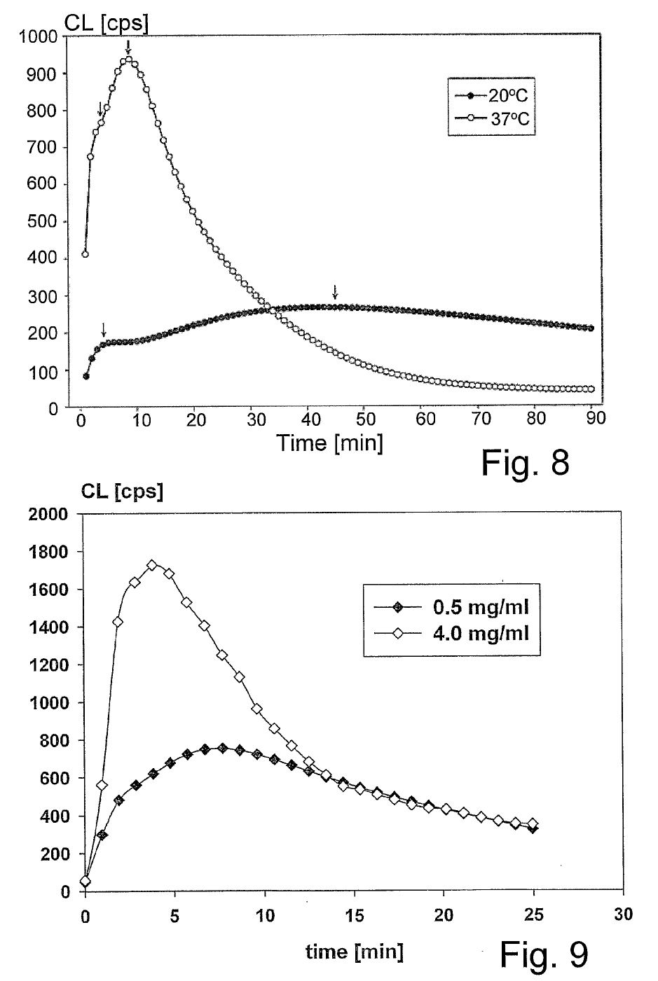

It has been observed that a silica surface may stimulate circulating blood phagocytes to produce a CL pattern similar to the first, extracellular, phase of the fMLP pattern [Tuomala et al.: Toxicol. Appl. Pharmacol. 118 (1993) 224-32]. Both the size of the target to be engulfed by phagocytes in this case, and the type of material (optic fiber) seem to significantly decrease the intracellular emission, and therefore the intracellular component is suppressed on the response curve. By applying the drop of blood on the end-face of fiber-optics, an increased surface-to-volume ratio is obtained, improving the conditions for phagocytosis, and furthermore, said missing intracellular component facilitates the chemiluminescent analysis by providing a distinct extracellular time-mark (see FIG. 8). This technical solution allows determining precisely the amounts of the extracellular part and intracellular part of phagocyte-generated ROS. As can be seen in FIG. 8, the temperature control is important.

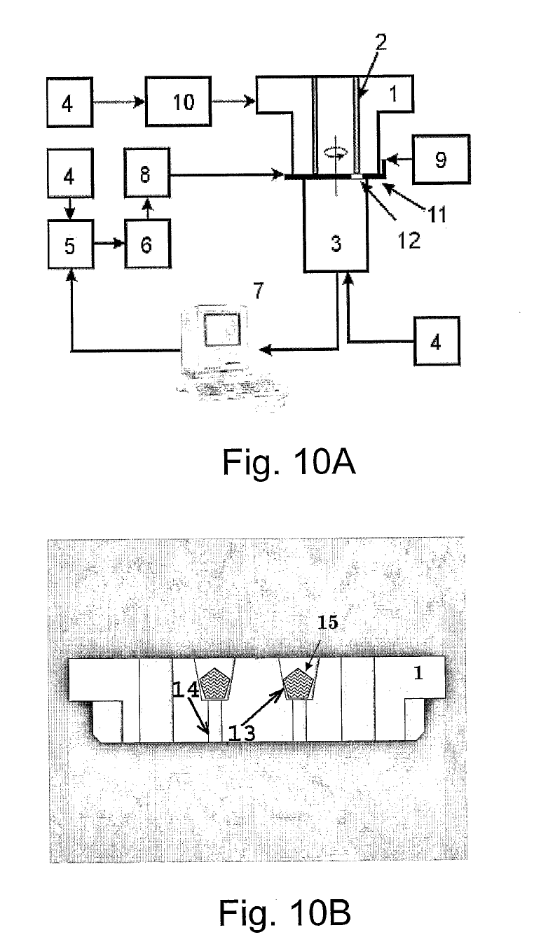

The invention is further directed to a device for evaluating phagocytes in a biological fluid provided by a patient, which device quantifies all the extracellular—and intracellular—parts of the chemiluminescence response simultaneously. A fiber-based luminometer according to the invention is a tool for rapid, sensitive, reproducible, and inexpensive measurement of the in vivo inflammation state of circulating phagocytes, and the evaluation of the patient status during infection. The luminometer comprises (a) computerized control of photodetection; (b) photon-counting mode measurement of multi-fiber-sample module; (c) simultaneous sending the measured data to a serial port (allowing for data acquisition by an external computer); (d) direct data record into the computer memory while placing the graphs in parallel on the computer screen; (e) printing of collected data. A block diagram of a multi-channel luminometer according to the invention is shown in FIG. 10A. It consists of a thermoregulated fiber holder module 1. As shown on FIG. 10B the thermoregulated fiber holder 1 module consists of a set of miniature cuvettes 13 for holding the tested sample 15, where said cuvettes 13 are integral part of the module body, and a set of standard optic fibers 14 (also shown in FIG. 10A-2). One end of the fiber 14 serves as the bottom of its corresponding cuvette 13, the other end shows at the bottom of the fiber holder module 1. Suitable fiber with an original Numerical Aperture (NA) of 0.22, can be obtained from multiple manufacturers, for example Fiberguide Industries, Stirling, USA. Their core is 1000 μm in diameter (refractive index of 1.457 at 633 nm) and it is surrounded by a 100 μm silica cladding (refractive index of 1.44 at 633 nm), followed by a 100 μm thick silicon buffer and finally a 100 μm thick black Tefzel® jacket. To remove most surface imperfections introduced by the fiber cleaving process and improve fiber optical geometry the fiber tips are polished using a step-down approach with polishing machine PLANPOL-2 (Struers) and diamond grinding pastes in sequence of 20 μm, 5 μm and then 1 μm (Sunva tools). Thermoregulation is achieved by thermocontact with a thermoregulated by a thermocontroller metal plate. Other parts are photon-counting PMT detector 3 (e.g., HC135-01, Hamamatsu); a DC power supply 4; a Programmable Logic Controller (PLC) 5 (e.g., SPC-10, Samsung); a stepper driver 6 (e.g. SD2, Digiplan); a personal computer 7 (e.g., Pentium/586); a step motor 8 (e.g., HY200-2220, Servo control Technology); a position sensor 9 (e.g., FS2-60, KEYENCE); a thermocontroller 10 (e.g., CT15, Minco); and a rotating disk-shutter 11. The rotating disk-shutter 11 is a non-transperant disk containing a hole 12 that is positioned under the sample-fiber 2 during the time in which it is under measurement, thereby exposing detector 3 to only one sample-fiber 2 at any given time.

The fiber holder (sample compartment 1) is designed to offer optimal light-capturing conditions for the adequate measurement of chemiluminescence emitted by the phagocytes lying at the end-face surface of the sample-fiber 2. The fiber holder 1 is designed for one-time use (i.e., disposable) and is disposed after the test. The light emission takes the place in a sample cuvette or well (13—shown in FIG. 10B). The disk-shutter 11 located in a light-tight space, can be rotated at corresponding angle around its axis by the step motor 8 and by a worm gear (not shown) with a preciseness of 0.025°. This rotation is controlled by PC 7 and instructions recorded in the memory of PLC 5 which are transmitted to the stepper motor 8 through stepper driver 6. When the orifice 12 of the rotating shutter 11 is positioned under one of the fibers 2 showing on the bottom end of the fiber-holder module 1, it is then in optical contact with the naked PMT head of the photon-counting detector 3 and the emitted photons are transmitted to the PMT surface and counted within a predetermined time interval. The subsequent turn of the shutter by a corresponding angle positions the next fiber for measurement and the cycle is thus repeated. The position sensor's feedback 9 is used to ensure the correct function of the shutter positioning. Neither the samples 15 (shown in FIG. 10B) nor the detector 3 change their position during the measurement. Such an arrangement is space thrifty, ensures constant thermo-regulation with the fiber holder 1, and provides a minimal optical path between the light-emitting samples 15 and the light detector 3, thus allowing optimum light collection. The measuring section consists of a photon-counting PMT detector 3 that responds to light emission with electric impulses, the number of which correlates with the number of photons emitted, i.e., light intensity.

The real CL kinetic data is modeled on the basis of equation. (1). The values of the component parameters are calculated using the iteration procedure to get the minimum sum of the squared differences between the real and the model CL intensity. The calculation is associated with boundary conditions for the time to maximal CL intensity of the corresponding components as follows:

1 component−T_{Imax}ε[1-3] min.

2 component−T_{Imax}ε[4-7] min.

3 component−T_{Imax}ε[>10] min.

Each component contributes to the total intensity depending on its own kinetics. This method of analysis can be implemented in a software application, designed to work with the said luminometer. The exact implementation of the software application is a standard task for software engineers. As explained above, the time values may differ, depending on the circumstances, but for any circumstances the three components may be identified, using the described analysis, and actualized times may be found. Another aspect of the invention is the disposable fiber holder 1 encapsulating number of sample-fibers 2. As it was mentioned earlier, the front-end surface of the silica optical fibers in our system also serves as an additional phagocyte stimulating agent always presenting in our light generating system. The optical fibers 14 are used as both light guides and cuvette bottom of sample holders 1. Indeed, both the size of the target to be phagocytized and the silica material will lead to one very important feature of the use of this device, the clear indication of the extracellularly produced light and its time appearance. The disposable part will also significantly facilitate the procedure of performing the tests by providing standard environments for phagocyte activation, and decreasing the efforts required for maintenance of the apparatus.

The invention will be further described and illustrated by the following examples.

### EXAMPLES

### Reagents

Zymosan-A (Sigma Chemical Co.) was used as a phagocyte-stimulating agent. It was opsonized for 30 min at 37° C. in sample serum (20 mg/mL) and washed twice in 0.9% NaCl. The zymosan suspension in Krebs-Ringer phosphate medium (KRP) was prepared immediately prior to use. KRP was composed of 119 mmol/L NaCl, 4.75 mmol/L KCl, 0.420 mmol/L CaCl₂, 1.19 mmol/L MgSO₄.7H2O, 16.6 mmol/L sodium phosphate buffer, pH 7.4 and 5.56 mmol/L glucose (De Sole et al., 1983). Luminol (Sigma Chemical Co.) was used to amplify the chemiluminescence activity. A luminol stock solution (10 mmol/L in dimethyl sulfoxide) was stored in a dark place at room temperature and diluted 1:10 (v/v) with KRP just before use. In all experiments, the final concentration of luminol was 100 μmol/L. In some experiments formylmethionyl-leucyl-phenylalanine (fMLP—Sigma Chemical Co.) was used for priming (5 nM) of CL emitting cells. All reagents used were of analytical grade and the water was glass-distilled.

### Chemiluminescence Assays

Diluted whole blood (1:100 v/v final dilution) was used to avoid artifacts due to the isolation of PMNs. Peripheral venous blood from human adults was collected in heparinized tubes (20 U/mL). Samples with a total volume of 200 μL contained diluted whole fresh blood, luminol and zymosan in KRP. The whole blood was diluted with KRP immediately prior to use. All reagents in the probe, with the exception of blood were pre-incubated in the luminometer at 37° C. for 5 min. After diluted blood was added, the sample content was mixed and CL measured.

LCL kinetics of six samples were simultaneously recorded using the previously described six-sample luminometer operating in photon counting mode. Each of the curves shown is representative of at least three experiments.

Three model LCL systems were investigated:

1. Standard system (S), containing 0.02 ml 1:10 diluted whole blood, 0.02 ml luminol (0.1 mmol/l) and different concentrations of zymosan in total volume of 0.2 ml. Blood was diluted with KRP immediately before use. The LCL kinetics of such a system includes both extra- and intracellularly generated light.
2. Primed system (P), contained the same reagents as the S, but prior to dilute the blood its phagocytes were primed using fMLP (5.0E-8M final concentration).
3. Aged system (A). Another test at S conditions was performed two hours after the “regular” S test. So the only difference between the consecutive runs was the “aged” blood.
4. Forced extracellular light emitting system (FES) (according to Magrisso M. et al.) [Biosens. Bioelectron. 21 (2006) 1210-18]), containing 6 mg/ml zymosan and optical fiber surface as an additional extra-cellular emission stimulating components of the above described standard system.

All sample compounds except blood were preincubated in the luminometer cuvettes at 37° C. for 5 minutes. After blood addition, the contents of the cuvettes were mixed, samples were put into the luminometer and registration of LCL kinetics was started. Two luminometers were used, Luminoskan Ascent, Thermo Labsystems, and a device according to the invention.

### Data Analysis

Various attitudes and possibilities were compared, when obtaining and analyzing data. For example, in order to estimate the phagocyte functional modification after a controlled priming in the whole blood using fMLP (5 min, 50 nM), two hours after the first standard assessment, a second measurement was performed under the same conditions (“aged” blood) in order to determine the time-derivatives of respiratory burst components. These triple-set of records were used for the subsequent CL component derivation and analysis.

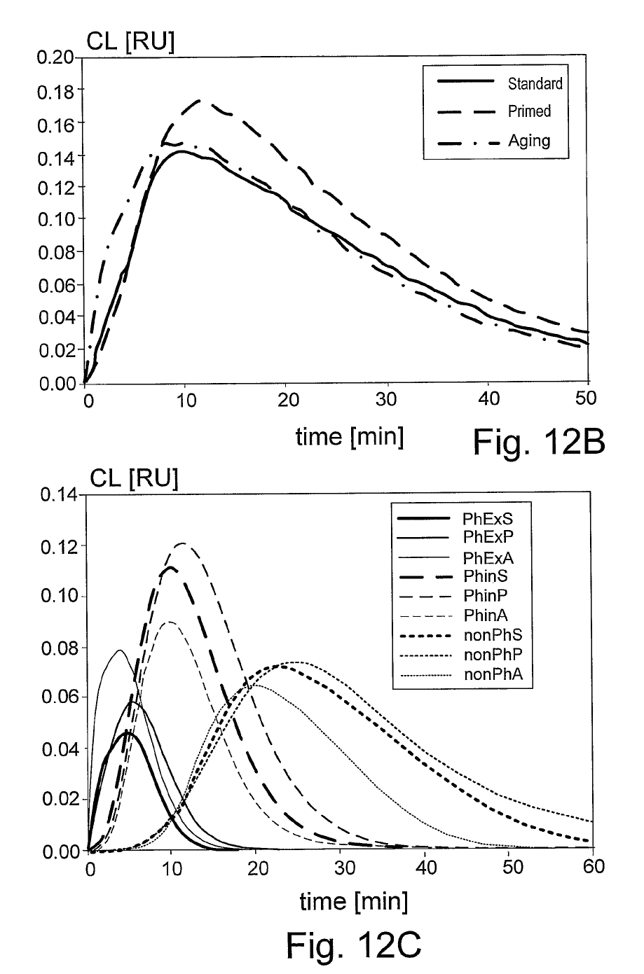

The general idea of dynamic component assessment of phagocyte respiratory burst is illustrated in FIGS. 1 and 12. The existing parameters of CL kinetics can be classified in three groups: physical, biological and temporal. The group of physical parameters consists of cell numbers (phagocytes, erythrocytes), stimulant concentration (particle/cell ratio), volume-to-surface ratio, mixing (sample oxygenation and phagocytosis synchronization), pH of the buffer used, and temperature. These parameters allow for calibration and the user of the method must keep them constant at some earlier predetermined value to avoid a multi-parametric interpretation. The other two groups of parameters may be more difficult to control, and therefore, the change in the phagocyte respiratory burst caused by some well controlled shift of these parameters may be rather employed. Relating to FIG. 11, the limited capacity of the phagocyte to restore its ability to generate ROS (inherent irreversibility of phagocytes), it is not the same if the phagocyte respiratory burst follows the S1-S0-S2′ or S1′-S0-S2 trajectory (both from an estimative and prognostic point of view). The quantification of every particular momentary state was performed by component analysis of CL kinetics. These “purposely shifted” CL kinetics were also quantified by component analysis and all data attributed to particular dynamic state of phagocytes were used to build a data base for future analysis.

In order to explore the relationships between the chemiluminescence data measuring phagocyte function and patients in different clinical conditions, several steps were performed. First, specific clinical and luminescent variables were recorded for all participating individuals (blind chemiluminescent measurements). Second, a full set of chemiluminescent data was derived by calculation and component analysis. Next, the patients with similar clinical status were placed into identifiable groups (such as healthy controls, dialysis patients without infection, dialysis diabetic without infection, dialysis with moderate infection, dialysis after transplantation). As a last step discriminant function analysis was used to determine which set of chemiluminescent variables discriminate between the occurring groups, to determine the canonical variables and canonical coefficients for every particular case.

Multiple discriminant analysis was earlier used [Stevens et al.: J. Infect. Dis. 170 (1994) 1463-72] to determine which variables discriminate between the groups of individuals with same diagnosis. The technique produces discriminant functions, which are linear combinations of the original variables. Next, the original variables were replaced by a new set of “canonical” variables in order to form two-dimensional graphic presentation of the data. These variables are constructed to show the greatest differences between the groups and are uncorrelated with each other. This is an effective form of data reduction that produces a set of variables that highlight the differences between the groups. For the purpose of this study, the following discriminant parameters were used in subsequent analysis: Capacity, Effectiveness, Velocity and background of respiratory burst derived by the component approach described earlier [Magrisso M. et al.: (2000) Ibid.], as well as other parameters non-related to phagocytosis and its localization. Other descriptive kinetic parameters may comprise initial slope, time to peak, etc. An important set of parameters is the one relating to respiratory burst and localization change due to some controlled “shift” in phagocyte activity, improving the phagocyte assessment. A list of useful parameters as well as their definitions is shown in Table 1.

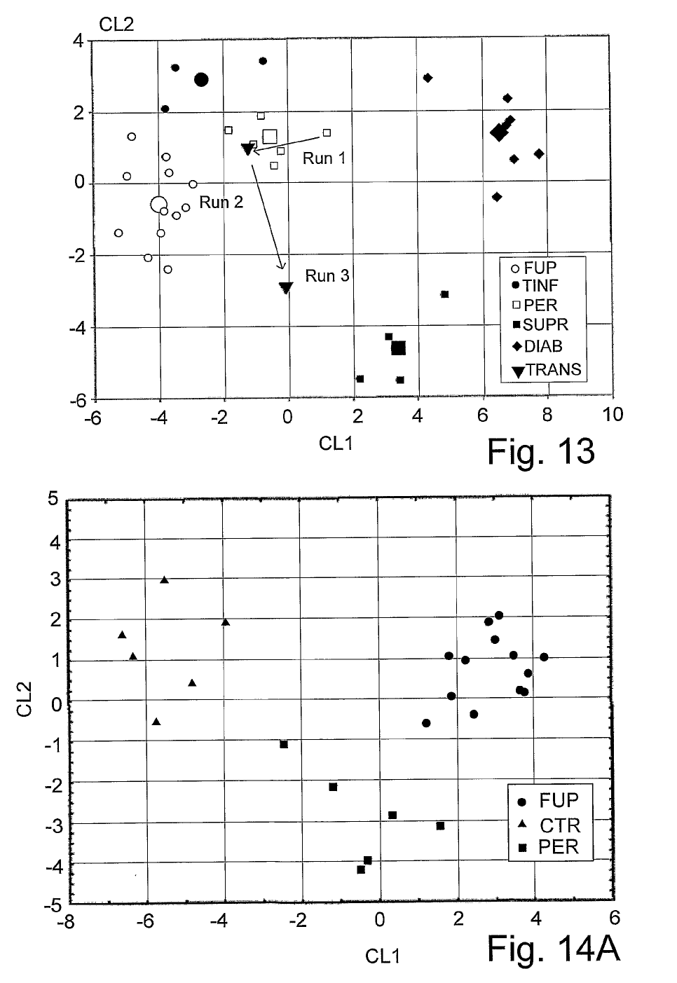

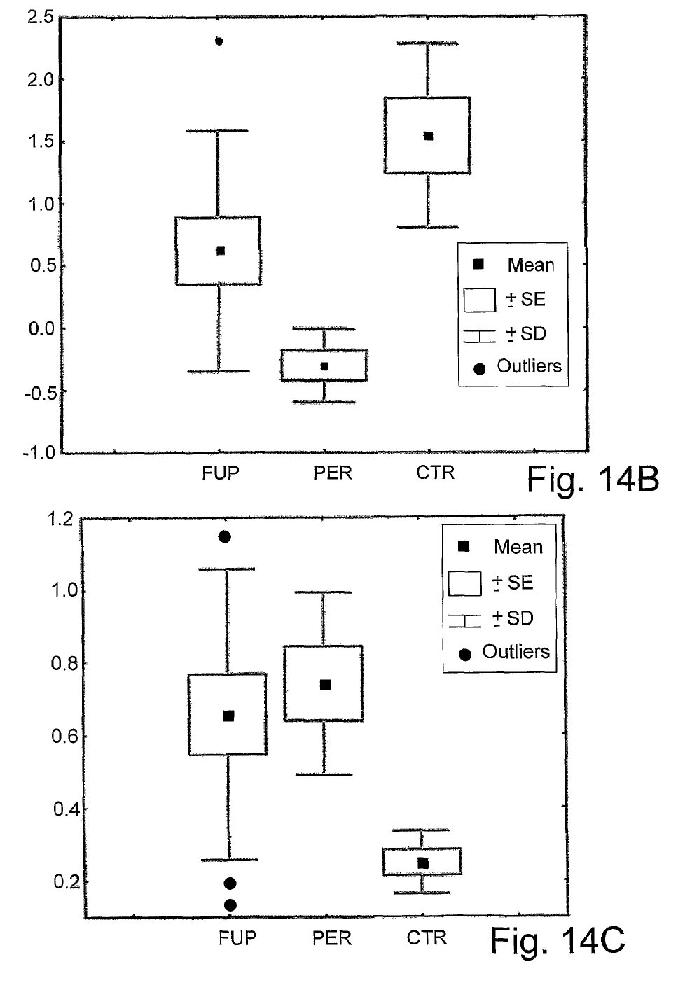

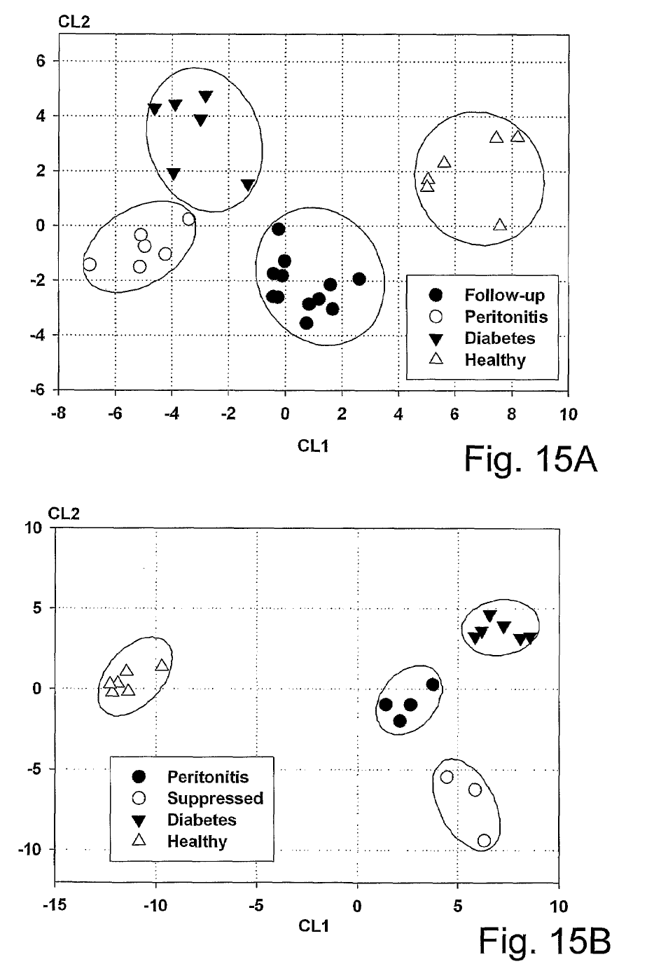

Linear discriminant analysis was used to calculate discriminant function coefficients for each patient group and to search for the relative contribution of each variable in discriminating between groups. These coefficients were then used to assess the probability that a given patient was correctly classified into a particular clinical group. For graphic presentations, a canonical analysis was used to reduce the number of dimensions to two. FIGS. 13 to 15 exemplify the use of said parameters.

Two types of relational analyses were done: daily-monitor studies of patients with specific infection and analysis of patients with different categories of CAPD population, such as follow-up, peritoneal infection, tunnel infection, suppressed-after-transplantation. In both types of analyses, data were compared to healthy (noninfected) controls.

TABLE 1
List of some parameters for group separation and case monitoring.

| Parameter | Definition |
|---|---|
| nonPhagoSA | Non-phago-related CL of aged sample |
| RelCapSP | Capacity of primed sample divided by capacity of |
| | standard sample |
| RelPtimeSP | Peak time of primed sample divided by peak time of |
| | standard sample |
| VelSP | Velocity of primed sample |
| ExtraS | Extra-cellular phagocytosis-related emission of |
| | standard sample |
| RelNoPhagoSA | Non-phago-related CL of aged sample divided by non- |
| | phago-related CL of standard sample |
| ExtraSA | Extra-cellular phagocytosis-related emission of primed |
| | sample |
| BkgSP | Background CL of primed sample |
| NoPhagoS | Non-phago-related CL of standard sample |
| VelSA | Velocity of aged sample |
| BkgSA | Background CL of aged sample |
| RelPtimeSA | Peak time of primed sample divided by peak time of aged |
| | sample |
| ExtraSP | Extra-cellular phagocytosis-related emission of primed |
| | sample |
| EffS | Effectiveness of standard sample |
| SlopeS | Peak of standard sample divided by time to reach it |
| SlopeSP | Peak of primed sample divided by time to reach it |

Using this procedure, the particular cases, based on clinical and chemiluminescent measurements for known homogenous groups, were classified. The phagocyte function in patients with infection or underlying diseases was subjected to the classification rule to calculate the most probable group membership. Sequential measurements during the illness were similarly analyzed and were used to track an individual's clinical course.

### Whole Blood Chemiluminescence

Cellular luminescence is dependent on erythrocyte number and is directly proportional to the number of phagocytes. Therefore, to normalize the CL results, the independent corrections of CL response to PMNL and RBC counts were applied to diluted whole blood samples after the record of CL kinetics [Bechev et al.: J. Bioche. Biophys. Methods 27 (1993) 301-9]. Several groups of patients were tested: healthy dialysis, healthy dialysis with diabetes, patients with peritonitis, tunnel infections suppressed after transplantation. FIGS. 13-15 comprise the different groups of patients.

### Patients Studied

Peritoneal dialysis is a method used to filter the blood when the kidneys do not work properly, involving passing a special fluid into the body's abdomen. The waste products pass from the blood, through a membrane lining the inside of the abdomen, into the special fluid, which can then be drained from the body. One type of peritoneal dialysis is continuous ambulatory peritoneal dialysis (CAPD). This does not require a machine, and it may be a possible approach for some mobile individuals. Healthy control subjects were laboratory personnel, medical students, or physicians who worked at the Soroka Medical Center in Beer Sheva, Israel. All control subjects were nonsmokers, were taking no prescription medications, had normal physical examination results, and had no acute illness during six weeks before the study. Subjects (54 patients, 6 controls) included in the groups were identified from patients attending an outpatient medical clinic. Patients who signed consent forms and ultimately had a specific diagnosis made were enrolled in the study. Specific diagnoses were based upon clinical findings, surgical operative findings, bacteriologic culture reports, and other laboratory results. No patients were excluded from the analysis. The mean ages of the healthy control group (53.2 years) and the patient populations (59.6 years) were not significantly different.

Samples of whole blood were removed from blood specimen obtained for routine complete blood cell and differential cell counts. This sample was immediately transported to the laboratory and assayed within 1 h as described. Total WBC and differential cell counts were determined in a clinical hematology laboratory. Patients were followed until resolution of infection or death. Additional assays were done sequentially during the course of infection, when the patient's clinical condition deteriorated, or before and after surgical intervention.

While the invention has been described using some specific examples, many modifications and variations are possible. It is therefore understood that the invention is not intended to be limited in any way, other than by the scope of the appended claims.

1. A method of assessing the in vivo dynamic state of phagocytes in a subject by measuring chemiluminescent (CL) kinetics resulting from reactive oxygen species (ROS) formation in vitro in a biological sample obtained from said subject and containing said phagocytes, said method comprising
i) dividing said sample to a plurality of portions;
ii) contacting the first portion of said sample with a chemiluminescent substrate and with a stimulating agent, and measuring a first CL signal, thereby obtaining a first kinetics;
iii) exposing the second portion of said sample to an agent or to conditions leading to a partial priming, and contacting said second portion with a chemiluminescent substrate and with a stimulating agent, and then measuring a second CL signal, thereby obtaining a second kinetics;
iv) optionally repeating step iii) for the third portion and for all other portions of said plurality of portions obtained by dividing said sample, thereby measuring a third and all other CL signals, constituting a plurality of signals, thereby obtaining a third kinetics and all other kinetics, constituting a plurality of kinetics;
v) analyzing said first kinetics, said second kinetics, and optionally said plurality of kinetics, comprising resolving each kinetics into at least three components (subkinetics) having maxima at least at three different times, the components corresponding to at least three different mechanisms of ROS formation; and
vi) calculating CL parameters, characterizing the kinetics and the subkinetics obtained with and without said priming agent or conditions, and characterizing the relationships between the kinetics.

2. The method of claim 1, wherein said subject exhibiting a certain diagnostic status is selected from the group consisting of a patient to be diagnosed, a healthy subject, a subject suffering from a defined medical condition, a subject undergoing a defined medical treatment, and a subject exposed to defined conditions affecting the dynamic state of phagocytes.

3. The method of claim 2, further comprising creating a database of standard values of said CL parameters, by employing steps i) to vi) of claim 1 on predetermined test groups of subjects, the subjects in each group exhibiting certain known diagnostic status, and by obtaining statistical characteristics of the measurements of each parameter for all subjects in each group, thereby obtaining a standard value of said parameter for said known diagnostic status.

4. The method of claim 2, further comprising comparing the CL parameters of said patient to be diagnosed with standard values obtained according to claim 3.

5. The method of claim 2, further comprising comparing the CL parameters of said patient to be diagnosed with known reference values, characteristic for said known diagnostic status.

6. The method of claim 1, wherein the stimulating agent is selected from the group consisting of optical fiber surface, opsonized zymosan, opsonized synthetic materials capable of fixing complement or eliciting specific antibody expression, opsonized attenuated bacteria, liquid stimulants, liquid stimulants, and combinations thereof.

7. The method of claim 1, wherein the biological sample comprises a diluted or undiluted biological fluid selected from the group consisting of whole blood, synovial fluid, amniotic fluid, cerebrospinal fluid, peritoneal fluid, pleural fluid, and pericardial fluid.

8. The method of claim 1, wherein said phagocytes are selected from the group consisting of neutrophils, monocytes, eosinophils, dendritic cells, and combinations thereof.

9. The method of claim 1, wherein said agent or conditions leading to a partial priming is selected from the group consisting of C5a, C5a.sub.desArg, N-formyl-methionyl peptides, leukotrienes, platelet activating factor, lipopolysaccharide, myeloid colony stimulating factors, cytokines, interferons, interleukins, chemokines, incubation (aging) at predetermined conditions, and combinations thereof.

10. The method of claim 1, wherein said agent or conditions leading to a partial priming enhance said second CL signal, and optionally also said plurality of signals, compared to said first CL signal, but under the conditions when said CL signals are lower than the maximally enhanced CL signal.

11. The method of claim 1, wherein said chemiluminescent substrate comprises luminol, isoluminol or lucigenin in solution.

12. The method of claim 1, wherein the CL light is monitored by a photometric instrument selected from the group consisting of a luminometer, a microscope photometer, and a fiber optic sensor.

13. The method of claim 1, wherein said three subkinetics correspond to three different mechanisms of ROS formation, the first of which comprises extracellular process related to phagocytosis, the second of which comprises an intracellular process related to phagocytosis, and the third of which comprises a process not directly connected with phagocytosis.

14. The method of claim 1, wherein said parameters are selected from the group consisting of total CL counts for a kinetics per phagocyte, total CL counts for a subkinetics per phagocyte, background CL counts, time of the maximal CL signal, Capacity (C), Effectiveness (E), and Velocity (V), and derivatives of the above parameters.

15. The method of claim 14, wherein said parameters relate to a stimulated sample, to a primed sample, to an aged sample, to a sample of said patient, to a control sample, or to their combinations.

16. The method of claim 1, wherein said analyzing comprises determining the contributions of intracellular and extracellular ROS forming processes.

17. The method of claim 3, wherein said standard values for a group of subjects exhibiting certain diagnostic condition are obtained by measuring chemiluminescent (CL) kinetics involved in the ROS formation in vitro in biological samples obtained from said subjects, said method comprising
i) dividing the sample obtained from a first subject to a plurality of portions;
ii) contacting the first portion of said first subject's sample with a chemiluminescent substrate and with a stimulating agent, and measuring a first CL signal, thereby obtaining a first kinetics;
iii) exposing the second portion of said first subject's sample to an agent or to conditions leading to a partial priming, and contacting said second portion with a chemiluminescent substrate and with a stimulating agent, and then measuring a second CL signal, thereby obtaining a second kinetics;
iv) optionally repeating step iii) for the third portion and for all other portions of said plurality of portions obtained by dividing said first subject's sample, thereby measuring a third and all other CL signals, constituting a plurality of signals, thereby obtaining a third kinetics and all other kinetics, constituting a plurality of kinetics, for said first subject;
v) analyzing said first kinetics, said second kinetics, and optionally said plurality of kinetics, for said first subject, comprising resolving each kinetics into at least three components having maxima at least at three different times (subkinetics), the components corresponding to at least three different mechanisms of ROS formation;
vi) calculating predetermined independent CL parameters characterizing the kinetics and subkinetics obtained with and without said priming agent, thereby obtaining a first measurement of said standard value for each independent CL parameter;
vii) repeating steps i) to vi) for samples obtained from a second, third, and all other subjects in said group of subjects exhibiting said diagnostic condition, thereby obtaining a second, third, and other measurements of said standard value; and
viii) calculating from said first, second, third, and all other measurements obtained in steps v) and vii), the mean value, confidence interval, and other statistical characteristics for each independent CL parameter, thereby obtaining the required standard value with the statistical characteristics of said CL parameter for said diagnostic condition.

18. The method of claim 17, wherein said predetermined independent parameters are selected so as to differentiate best, in a statistically significant manner, between two or more groups of subjects exhibiting different diagnostic conditions.

19. The method of claim 17, wherein said independent parameters are selected by using multiple discriminant analysis.

20. The method of claim 2, wherein said medical condition is selected from the group consisting of infection, inflammation, immunity disorder, and stress or trauma related disorder.

21. The method of claim 1, comprising assessing the in vivo functional state of phagocytes in a human or animal patient by determining the normalized amounts and proportions of extracellularly and intracellularly generated ROS during interactions of said phagocytes contained in a biological sample with a stimulating agent, comprising
i) determining the approximate number of phagocytes and erythrocytes in said sample;
ii) determining the normalized extents of extracellularly and intracellularly phagocytes-generated ROS over a predetermined time period in a first portion of said sample;
iii) determining the extents of extracellularly and intracellularly phagocytes-generated ROS over said time period in a second, and optionally in a third portion and in other portions of said sample, which second portion and other portions were exposed to an agent or conditions causing a partial priming which shifted the functional state of the phagocytes in said samples, wherein said priming agents and conditions are different in all portions;
iv) comparing the extents of extracellularly and intracellularly phagocytes-generated ROS over said time period and their proportions of said first portion, with the extents and their proportions of said second portion, and optionally also of said third and other portions, of the sample, obtaining parameters reflecting said functional state of phagocytes; and
v) comparing said parameters obtained in step iv) with a range of controls, enabling to assess the functional state of the phagocytes.

22. A method of measuring chemiluminescent (CL) kinetics resulting from reactive oxygen species (ROS) formation in vitro in a biological sample containing phagocytes, comprising
i) dividing said sample to a plurality of portions;
ii) contacting the first portion of said sample with a chemiluminescent substrate and with a stimulating agent, and measuring a first CL signal, thereby obtaining a first kinetics;
iii) exposing the second portion of said sample to conditions leading to a partial priming or to an agent leading to a partial priming, and contacting said second portion with a chemiluminescent substrate and with a stimulating agent, and then measuring a second CL signal, thereby obtaining a second kinetics; wherein said stimulating agent in steps ii) and iii) and said agent leading to a partial priming (priming agents) are either standard agents or tested agents;
iv) optionally repeating step iii) for the third portion and for all other portions of said plurality of portions obtained by dividing said sample, thereby measuring a third and all other CL signals, constituting a plurality of signals, thereby obtaining a third kinetics and all other kinetics, constituting a plurality of kinetics;
v) analyzing said first kinetics, said second kinetics, and optionally said plurality of kinetics, comprising resolving each kinetics into at least three components having maxima at least at three different times (subkinetics), the components corresponding to at least three different mechanisms of ROS formation;
vi) calculating CL parameters, characterizing the kinetics and the subkinetics obtained with and without said priming agent, and characterizing the relationships between the kinetics; and
vii) comparing the CL parameters obtained in steps i) to vi) for standard agents with the CL parameters obtained in the same steps for tested agents; wherein standard agents are any agents whose effect on the phagocytes is known, and the tested agents are agents whose effect of the phagocytes is examined.

23. The method of claim 22, wherein said standard stimulating agent is selected from the group consisting of optical fiber surface, opsonized zymosan, opsonized synthetic materials capable of fixing complement or eliciting specific antibody expression, opsonized attenuated bacteria, liquid stimulants, and combinations thereof.

24. The method of claim 22, wherein said priming agent is selected from the group consisting of C5a, C5a.sub.desArg, N-formyl-methionyl peptides, leukotrienes, latelet activating factor, lipopolysaccharide, myeloid colony stimulating factors, cytokines, interferons, interleukins, chemokines, incubation (aging) at predetermined conditions, and combinations thereof.

25. A method for testing an effect of a pharmacologically important agent (tested agent) on phagocytes by analyzing in vitro interactions between said agent and said phagocytes, including measuring chemiluminescent (CL) kinetics according to claim 22, comprising:
i) providing a sample containing phagocytes, and determining the approximate number of phagocytes and erythrocytes in the sample;
ii) contacting a first portion of said sample with a standard stimulating agent and with a chemiluminescent substrate, optionally contacting said first portion with a standard priming agent before said contacting with the stimulating agent and the chemiluminescent substrate, and measuring a first CL kinetics;
iii) determining the amounts of extracellularly and intracellularly phagocytes-generated ROS over a predetermined time period in said first portion;
iv) contacting a second portion of said sample with a stimulating agent and, when in step ii) a priming agent was used, with a priming agent, wherein at least one of said stimulating agent and said priming agent is said tested agent—the other one being said standard agent, followed by contacting with the chemiluminescent substrate, and measuring a second CL kinetics;
v) determining the amounts of extracellularly and intracellularly phagocytes-generated ROS over a predetermined time period in said second portion;
vi) comparing the amounts and proportions of extracellularly and intracellularly phagocyte-generated ROS of the first and the second portions of the sample, thereby obtaining the in vitro effect of the tested agent on the phagocytes; optionally
vii) comparing said second CL kinetics with CL kinetics corresponding to a range of control phagocytes-samples obtained from patients exhibiting a range of diagnostic conditions, thereby comparing the effect of said tested agent with an effect of various diagnostic conditions on the phagocytes; and optionally
viii) comparing said second CL kinetics with CL kinetics corresponding to a range of control phagocytes-samples treated according to steps
i) to vi), wherein instead of said tested agent one or a plurality of other pharmacologically important agents were used, whose effect on the phagocytes is known, thereby comparing the effect of said tested agent with an effect of various other pharmacologically important agents.

26. The method of claim 25, wherein said phagocytes are selected from the group consisting of neutrophils, monocytes, eosinophils, dendritic cells, and combinations thereof.

27. The method of claim 25, wherein the tested agent is selected from the group consisting of metals, ceramics, bioresorbables, breakdown products of bioresorbables, hydroxyapatite, polyglycolic acids, nylon, silk, polymers, polyactic acids, glutaraldehyde, modified natural and synthetic materials, and combinations thereof.

28. The method of claim 25, wherein the tested agent is selected from the group consisting of therapeutic and pharmaceutical agents, and combinations thereof.

29. The method of claim 25, wherein the tested agent is selected from the group consisting of cytotoxic agents.

30. An apparatus for determining the in vivo dynamic functional state of phagocytes in a subject, comprising
i) at least one sensor for measuring a CL kinetics in a biological sample containing phagocytes in contact with a stimulating agent, and optionally with a priming agent, and with a CL substrate; and
ii) a processor for resolving said CL kinetics into at least three subkinetics corresponding to at least three different mechanisms of ROS formation.

31. The apparatus of claim 30, measuring simultaneously or consequently two CL kinetics in at least two portions of one sample, wherein the two portions differ in the concentrations of said stimulating and/or priming agents.

32. The apparatus of claim 30, measuring a plurality of portions divided from one sample.

33. The apparatus of claim 30, measuring a plurality of samples obtained from plurality of subjects.

34. The apparatus of claim 30, wherein said processor
i) receives from said sensor a signal corresponding to at least two different kinetics, and resolves each of the kinetics into at least three subkinetics;
ii) calculates CL parameters characterizing the kinetics and subkinetics and their relations;
iii) compares said CL parameters with standard values of said parameters, stored in the memory, corresponding to a range of diagnostic conditions; and
iv) provides an assessment of the in vivo dynamic state of the patient's phagocytes.

35. The apparatus of claim 34, wherein said sensor comprises an optical fiber that is in direct contact with said sample containing phagocytes.

36. The apparatus of claim 34 for determining a functional state of phagocytes of a subject, comprising
i) a sensor for single or multiple measurements of CL kinetics involved in generating ROS over a predetermined time period in a phagocyte-containing biological sample of a patient; and
ii) a processor for determining the extent of extracellularly and intracellularly generated ROS.

37. The apparatus of claim 36, comprising a sample compartment, temperature control, measuring compartment, optical fiber, and photodetector.

38. The apparatus of claim 37, wherein the end-face of said optical fiber is integrated into the wall of said compartment.

39. The apparatus of claim 37, wherein said end-face of the optical fiber serves as a phagocytosis stimulator.

40. The apparatus of claim 37, wherein said photodetector can measure the incident light in a photon-counting mode.

41. The apparatus of claim 36, wherein the phagocyte functional state measurements is performed automatically.

42. The apparatus of claim 36, wherein the phagocyte dynamic functional state measurements are performed without changing the sample and detector position.

43. The apparatus of claim 36, wherein said measurement is performed on more than one blood sample.

44. The apparatus of claim 43, wherein said measurement is performed on a native blood sample, and on an exogenously stimulated blood sample.

45. The apparatus of claim 30, wherein said subkinetics are approximated by Poisson distribution curves.

46. A kit for use in the evaluation of the in vivo dynamic state of phagocytes, of a patient, comprising:
i) disposable chamber(s) or parts thereof in which measuring CL kinetics occurs, which kinetics are involved in the ROS formation in a biological sample obtained from said patient containing said phagocytes;
ii) an opsonized, oxidative metabolism stimulating, agent;
iii) a chemiluminescent (chemiluminigenic) substrate; and
iv) a priming agent in an amount sufficient to obtain phagocytes with a shifted functional state in a portion of said sample, but in an amount lower than amounts eliciting maximal response.

47. The kit of claim 46 for use in the apparatus of claim 31.

48. The kit of claim 45, wherein said disposable chamber or its part comprises chamber surface, chamber surface with bound stimulating agent, chamber surface with bound CL substrate, or combinations thereof.

49. The kit of claim 46, wherein the surface of said chamber is selected from the group consisting of optical fiber surface, glass surface, surface stimulating phagocytes, surface stimulating extracellularly formed CL, and combinations thereof.

50. The kit of claim 48, wherein said bound materials are selected from the group consisting of receptor stimulants, non-receptor stimulants, opsonized zymosan, opsonized synthetic materials capable of fixing complement, materials eliciting specific antibody expression, opsonized attenuated bacteria, and combinations thereof.

51. The kit of claim 48, wherein said bound materials are selected from the group consisting of luminol, isoluminol, and lucigenin.

52. The kit of claim 46, wherein said priming agent is selected from the group consisting of C5a, C5a.sub.desArg, N-formyl-methionyl peptides, leukotrienes, latelet activating factor, lipopolysaccharide, myeloid colony stimulating factors, cytokines, interferons, interleukins, chemokines, and combinations thereof.
